(19) United States
(12) Patent Application Publication
(10) Pub. No.: US 2025/0299824 A1
(43) Pub. Date: Sep. 25, 2025

(54) MACHINE LEARNING ENABLED ANALYSIS OF OPTICAL COHERENCE TOMOGRAPHY ANGIOGRAPHY SCANS FOR DIAGNOSIS AND TREATMENT

(71) Applicant: Genentech, Inc., South San Francisco, CA (US)

(72) Inventor: Acner Camino BENECH, South San Francisco, CA (US)

(21) Appl. No.: 19/230,439

(22) Filed: Jun. 6, 2025

Related U.S. Application Data
(63) Continuation of application No. PCT/US2023/084150, filed on Dec. 14, 2023.
(60) Provisional application No. 63/387,464, filed on Dec. 14, 2022.
(60) Provisional application No. 63/494,416, filed on Apr. 5, 2023.

(51) Int. Cl. G16H 50/20; G06T 7/00

(52) U.S. Cl. CPC G16H 50/20; G06T 7/0012; G06T 2207/10101; G06T 2207/20081; G06T 2207/20084

(57) ABSTRACT

Methods and systems for foveal avascular zone (FAZ) segmentation of a retina. A three dimensional optical coherence tomography angiography (OCTA) volume of a retina of a subject is received. The OCTA volume comprising a plurality of layers. A slab input for a model system is formed using the OCTA volume. The model system comprising a deep learning model. A set of mask images is generated, via the model system, based on the slab input. The set of mask images includes an area mask image that accurately and reliably identifies an area of a foveal avascular zone captured by the OCT volume.

### CROSS-REFERENCE TO RELATED APPLICATIONS

This application is a continuation of International Application No. PCT/US2023/084150, filed on Dec. 14, 2023, which is related to and claims the benefit of the priority date of U.S. Provisional Application 63/494,416, filed Apr. 5, 2023, entitled “Machine Learning Enabled Analysis of Optical Coherence Tomography Angiography Scans for Diagnosis and Treatment,” as well as of U.S. Provisional Application 63/387,464, filed Dec. 14, 2022, entitled “Machine Learning Enabled Analysis of Optical Coherence Tomography Angiography Scans for Diagnosis and Treatment,” each of which is incorporated herein by reference in its entirety.

### TECHNICAL FIELD

The present disclosure relates generally to analyzing optical coherence tomography angiography (OCTA) scans and, more specifically, to analyzing OCTA scans using machine learning to provide indications about the prognosis of retinal disease.

### BACKGROUND

Ocular imaging includes a variety of imaging modalities capable of providing real-time, non-invasive, and high-resolution images of the eye. Retinal imaging captures digital images of the anatomical features present within the interior of the eye including, for example, the retina, optic nerve head, blood vessels, and/or the like. Optical coherence tomography (OCT) and optical coherence tomography angiography (OCTA or OCT-A) are two examples of retinal imaging modalities. While optical coherence tomography (OCT) uses light waves to capture cross sectional images showing the various layers of the retina, optical coherence tomography angiography (OCTA) performs repeated optical coherence tomography (OCT) acquisitions at a same tissue location in order to generate volumetric angiography images that depict the microvascular structure of the retina. Analyses of such OCTA images, which may include a large amount of data, are performed manually, and usually by subject matter experts, and as such can be cumbersome and very expensive. Thus, it may be desirable to have methods and systems that facilitate the consistent, accurate, and quick analyses of large amounts of medical images such as OCTA images for use in the diagnosis, monitoring, and treatment of patients.

### SUMMARY

In one or more embodiments, a method for segmentation of a retina is provided. A three dimensional optical coherence tomography angiography (OCTA) volume of a retina of a subject is received. The OCTA volume comprising a plurality of layers. A slab input for a model system is formed using the OCTA volume. The model system comprising a deep learning model. A set of mask images is generated, via the model system, based on the slab input. The set of mask images includes an area mask image that accurately and reliably identifies an area of a foveal avascular zone captured by the OCT volume.

In one or more embodiments, a method for training a model system is provided. A training dataset that that includes a plurality of optical coherence tomography angiography (OCTA) volumes for a plurality of retinas is received. A set of augmentation operations is performed to form a 2D image input that includes a plurality of 2D images. A model system is trained to generate a set of mask images that includes an area mask image that identifies an area of a foveal avascular zone using the two-dimensional training image input.

In one or more embodiments, a system comprises at least one data processor; and at least one memory storing instructions, which when executed by the at least one data processor, cause the processor to receive a training dataset that includes a plurality of optical coherence tomography angiography (OCTA) volumes for a plurality of retinas; and train a model system using a loss function over a plurality of training cycles to generate a set of mask images that includes an area mask image that identifies an area of a foveal avascular zone based on a slab input. The model system comprises a foveal avascular zone module, a foveal avascular zone boundary module, and a vessel module. The loss function includes at least one loss metric for each of the foveal avascular zone module, the foveal avascular zone boundary module, and the vessel module.

In one or more embodiments, a system comprises at least one data processor; and at least one memory storing instructions, which when executed by the at least one data processor, result in operations comprising any one or more of the methods described herein or a portion thereof.

In one or more embodiments, a non-transitory computer readable medium storing instructions is provided, which when executed by at least one data processor, result in comprising any one or more of the methods described herein or a portion thereof.

### BRIEF DESCRIPTION OF THE DRAWINGS

The accompanying drawings, which are incorporated in and constitute a part of this specification, show certain aspects of the subject matter disclosed herein and, together with the description, help explain some of the principles associated with the disclosed implementations. In the drawings:

FIG. 1 is a block diagram of an optical coherence tomography angiography (OCTA) image processing system 100 in accordance with one or more example embodiments.

FIG. 2 is a block diagram of the foveal avascular zone (FAZ) analysis system from FIG. 1 described in further detail with respect to a training mode in accordance with one or more embodiments.

FIG. 3 is a block diagram of the foveal avascular zone (FAZ) analysis system from FIGS. 1-2 described in further detail in accordance with one or more embodiments.

FIG. 4 is a schematic diagram illustrating the creation of a two-dimensional image from an OCTA volume in accordance with one or more embodiments.

FIG. 5 is a schematic diagram of a workflow for generating a set of mask images in accordance with one or more embodiments.

FIG. 6 is a flowchart of a process for analyzing an OCTA volume of a retina of a subject in accordance with one or more example embodiments.

FIG. 7 is a flowchart of a process for training a model system to generate a set of mask images in accordance with one or more embodiments.

FIG. 8 is flowchart of a process for performing a set of augmentation operations in accordance with one or more example embodiments.

FIG. 9 is flowchart of a process for computing a loss based on a set of mask images and training mask images using a loss function in accordance with one or more example embodiments.

FIGS. 10A-10C depict examples of two-dimensional projections of slabs of three-dimensional optical coherence tomography angiography (OCTA) volumes exhibiting different disease observations associated with diabetic retinopathy in accordance with one or more embodiments.

FIGS. 11A-11D depict examples of different types of augmentation that may be performed as part of training a model system in accordance with one or more example embodiments.

FIGS. 12A-12C depict examples of two-dimensional projections of slabs of OCTA volumes produced by three different imaging systems (e.g., OCTA scanners) in accordance with one or more embodiments.

FIG. 13 depicts a comparison of training a model system in two different ways in accordance with one or more embodiments.

FIG. 14 is a block diagram illustrating an example of a computing system, in accordance with one or more example embodiments.

When practical, similar reference numbers denote similar structures, features, or elements. It is to be understood that the figures are not necessarily drawn to scale, nor are the objects in the figures necessarily drawn to scale in relationship to one another. The figures are depictions that are intended to bring clarity and understanding to various embodiments of apparatuses, systems, and methods disclosed herein. Wherever possible, the same reference numbers will be used throughout the drawings to refer to the same or like parts. Moreover, it should be appreciated that the drawings are not intended to limit the scope of the present teachings in any way.

### DETAILED DESCRIPTION

### I. Overview

The embodiments described herein recognize and take into account that medical imaging technologies are powerful tools that can be used to produce medical images that allow healthcare practitioners to better visualize and understand the medical issues of their patients, and as such provide the same more accurate diagnoses and treatment options. The embodiments described herein recognize that the foveal avascular zone, which is a certain part of the retina, may provide valuable information that can be used to accurately diagnose and treat retinal diseases, and monitor the retina more generally.

One such retinal disease includes diabetic retinopathy (DR), a common complication of chronic diabetes that can lead to vision loss if not adequately treated. Diabetic retinopathy (DR) is a common microvascular complication in subjects with diabetes mellitus. DR occurs when high blood sugar levels cause damage to blood vessels in the retina. The two stages of DR include the earlier stage, non-proliferative diabetic retinopathy (NPDR), and the more advanced stage, proliferative diabetic retinopathy (PDR). With NPDR, tiny blood vessels may leak and cause the retina and/or macula to swell. In some cases, macular ischemia may occur, tiny exudates may form in the retina, or both. With PDR, new, fragile blood vessels may grow in a manner that can leak blood into the vitreous humor, damage the optic nerve, or both. Untreated, PDR can lead to severe vision loss and even blindness.

The embodiments described herein recognize that DR progression can be monitored using optical coherence tomography angiography (OCTA), a non-invasive imaging technique that can be used to identify key features of DR, such as changes in foveal vascular density and foveal avascular zone enlargement.

With OCTA, multiple optical coherence tomography (OCT) acquisitions are performed at a same tissue location to detect differences in the light scatter behavior of blood, capillaries, and large vessels that relate to the motion produced by blood flow in the retinal and choroidal microvasculature. For example, blood can exhibit a high probability of engendering multiple scattered light paths due to its a high scattering anisotropy (or property of being directionally dependent). Meanwhile, the movement of red blood cells through capillaries can produce forward scattering that precedes or follows static tissue backscattering (e.g., “multiple scattering” tails). In larger vessels where the backscattering cross-section is determined by the shear-induced orientation of red blood cells with their flat face parallel to the shear force, multiple intravascular dynamic scattering events are likely to be observed if the vessel lumen exceeds a scattering length. Accordingly, the resulting volumetric angiography image is a three-dimensional volumetric image in which each pixel value may be positively correlated with the presence of vessel in the retina.

Compared to existing angiography techniques such as fluorescein angiography (FA) and indocyanine green angiography (ICGA), optical coherence tomography angiography (OCTA) is capable of capturing the microvascular structure of the retina in greater detail, thus enabling the detection of more obscure conditions such as irregular foveal avascular zone (FAZ), capillary non-perfusion, intraretinal microvascular abnormalities, and/or the like.

Analysis of optical coherence tomography angiography (OCTA) scans may be performed on a two-dimensional projection of the corresponding three-dimensional volumetric angiography image. Instead of the longitudinal cross-sectional images associated with optical coherence tomography (OCT), the two-dimensional projection of a three-dimensional optical coherence tomography angiography (OCTA) volume may be an en-face projection depicting a transverse view of the retina at various depths. Analysis of the two-dimensional projection of the three-dimensional optical coherence tomography angiography (OCTA) volume may reveal insights into a variety of retinal diseases. For example, various characteristics of a retina's microvascular structure observed in the two-dimensional projection of a three-dimensional optical coherence tomography angiography (OCTA) scan, including that of the foveal avascular zone and the surrounding vessels, may be indicative of the disease burden and disease progression of diabetic retinopathy as well as the efficacy of treatments. Nevertheless, in certain cases, conventional analytical techniques may be error prone due to a high level of inter- and intra-pathologist variability.

Currently, FAZ measurements can be automated using various OCTA device software, a process that is faster than manual FAZ segmentation. However, currently available systems and methods for performing these automations are not always reliable when conducted on retinas affected by retinal diseases such as DR. Accordingly the segmentation results of these automations may require manual correction by human graders. Manual corrections can be time consuming, costly, undesirable, and even unfeasible in large datasets. Further, manual corrections may not have the level of accuracy that is desired. For example, significant inter-clinician and intra-clinician variability can be present in subsequent correction efforts.

Thus, the embodiments described herein recognize that it desirable to have improved methods and systems for performing segmentation of the foveal avascular zone in an OCTA volume accurately, precisely, and reliably. The embodiments described herein provide methods and systems for accurately, precisely, and/or reliably performing FAZ segmentation using a model trained based on the identification of an area of the foveal avascular zone as well as the boundary of and/or vessels associated with the foveal avascular zone. The vessels associated with the foveal avascular zone are those vessels outside the foveal avascular zone. The embodiments described herein provide methods and systems that may reduce the overall computing resources and time that would be needed to otherwise perform such FAZ segmentation.

Further, with more accurate FAZ segmentation, more accurate measurements relating to the foveal avascular zone may be computed. More accurate measurements relating to the foveal avascular zone may result in the ability to provide more accurate and reliable indications for the prognosis of a retina with respect to retinal disease (e.g., DR). For example, FAZ segmentation may be used to generate one or more measurements including, for example, but not limited to, a measurement for the area of the foveal avascular zone, a measurement indicating a circularity of the foveal avascular zone, a measurement indicating a tortuosity of the foveal avascular zone, vessel density, perfusion density, fractal dimension, vessel tortuosity index, vessel caliber index, or one or more other measurements.

### II. Example System for FAZ Area Segmentation

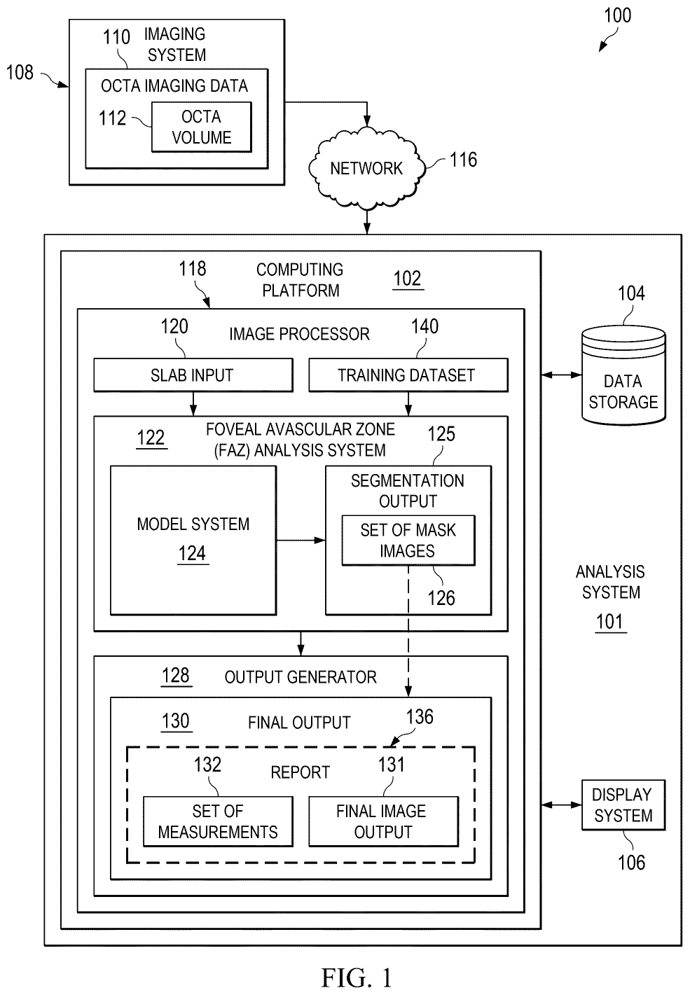

FIG. 1 is a block diagram of an optical coherence tomography angiography (OCTA) image processing system 100 in accordance with one or more example embodiments. Image processing system 100 may be used to process ophthalmological images to extract features from such images, correct or otherwise adjust one or more feature extracted from such images, segment such images, generate one or more outputs related to the diagnosis, screening, and/or treatment of an ophthalmological disorder, or a combination thereof.

The image processing system 100 includes analysis system 101. Analysis system 101 may be implemented using hardware, software, firmware, or a combination thereof. In one or more embodiments, analysis system 101 may include a computing platform 102, a data storage 104 (e.g., database, server, storage module, cloud storage, etc.), and a display system 106. Computing platform 102 may take various forms. In one or more embodiments, computing platform 102 includes a single computer (or computer system) or multiple computers in communication with each other. In other examples, computing platform 102 takes the form of a cloud computing platform, a mobile computing platform (e.g., laptop, a smartphone, a tablet, etc.), another processor-based device (e.g., a workstation or desktop computer) or a wearable computing device (e.g., a smartwatch), and/or the like or a combination thereof.

Data storage 104 and display system 106 are each in communication with computing platform 102. In some examples, data storage 104, display system 106, or both may be considered part of or otherwise integrated with computing platform 102. Thus, in some examples, computing platform 102, data storage 104, and display system 106 may be separate components in communication with each other, but in other examples, some combination of these components may be integrated together.

Computing platform 102 may be or may be part of a client device that is a processor-based device including, for example, a workstation, a desktop computer, a laptop computer, a smartphone, a tablet computer, a wearable apparatus, and/or the like.

The imaging processing system 100 may further include imaging system 108. In one or more embodiments, imaging system 108 includes an optical coherence tomography angiography (OCTA) system (e.g., OCTA scanner or machine) that is configured to generate OCTA imaging data 110 for the tissue of a patient. The imaging system 108 may include one or OCTA scanners including, for example, a swept-source scanner, a spectral domain scanner, and/or other types of scanners. In some instances, imaging system 108 can be a large tabletop configuration used in clinical settings, a portable or handheld dedicated system, or a “smart” OCT system incorporated into user personal devices such as smartphones.

OCTA imaging data 110 may include any number of three-dimensional, two-dimensional, or one-dimensional spectral domain (SD) optical coherence tomography (OCT) images. OCTA imaging data 110 may include OCTA volume 112. OCTA volume 112 may be generated by performing repeated OCT scans at a same tissue location. Each of those scans may form a layer in OCTA volume 112. Accordingly, OCTA volume 112 may include a plurality of OCTA layers.

According to some embodiments, imaging system 108 may be used to generate OCTA imaging data 110 for a retina of a patient. In some embodiments, the retina is a healthy retina. In other embodiments, the retina is one that has been diagnosed with a retinal disease. For example, the diagnosis may be one of age-related macular degeneration (AMD), neovascular age-related macular degeneration (nAMD), diabetic retinopathy (DR), macular edema, geographic atrophy, or some other type of retinal disease.

Analysis system 101 may be in communication with imaging system 108 via network 116. Network 116 may be implemented using a single network or multiple networks in combination. Network 116 may be implemented using any number of wired communications links, wireless communications links, optical communications links, or combination thereof. For example, in various embodiments, network 116 may include the Internet or one or more intranets, landline networks, wireless networks, and/or other appropriate types of networks. In another example, the network 116 may include a wireless telecommunications network (e.g., cellular phone network) adapted to communicate with other communication networks, such as the Internet. In some cases, network 116 includes at least one of a local area network (LAN), a virtual local area network (VLAN), a wide area network (WAN), a public land mobile network (PLMN), the Internet, or another type of network.

The imaging system 108 and analysis system 101 may each include one or more electronic processors, electronic memories, and other appropriate electronic components for executing instructions such as program code and/or data stored on one or more computer readable mediums to implement the various applications, data, and steps described herein. For example, such instructions may be stored in one or more computer readable media such as memories or data storage devices (e.g., data storage 104) internal and/or external to various components of image processing system 100, and/or accessible over network 116.

Although only one of each of imaging system 108 and the analysis system 101 is shown, there can be more than one of each in other embodiments. Further, although FIG. 1 shows the imaging system 108 and the analysis system 101 as two separate components, in some embodiments, the imaging system 108 and the analysis system 101 may be parts of the same system (e.g., maintained by the same entity such as a health care provider or clinical trial administrator). In some cases, a portion of analysis system 101 may be implemented as part of imaging system 108. For example, analysis system 101 may be configured to run as a module implemented using a processor, microprocessor, or some other hardware component of imaging system 108. In still other embodiments, analysis system 101 may be implemented within a cloud computing system that can be accessed by or otherwise communicate with imaging system 108.

Analysis system 101 may include an image processor 118 that is configured to receive OCTA imaging data 110 from the imaging system 108. The image processor 118 may be implemented using hardware, firmware, software, or a combination thereof. In one or more embodiments, image processor 118 may be implemented within computing platform 102. In some cases, at least a portion of (e.g., a module of) image processor 118 is implemented within imaging system 108.

The image processor 118 may include a foveal vascular zone (FAZ) analysis system 122 for processing OCTA imaging data 110. For example, FAZ analysis system 122 may form slab input 120 that is to be input into model system 124 of FAZ analysis system 122 based on OCTA imaging data 110. In one or more embodiments, OCTA imaging data 110 includes slab input 120. In other embodiments, FAZ analysis system 122 identifies a portion of the layers that make up OCTA volume 114 as the slab input 120.

Slab input 120 includes one or more slabs. As used herein, a slab is a three-dimensional portion of an OCTA volume. A slab may correspond to, for example, a plexus of the retina including, for example, a retinal nerve fiber layer plexus, a superficial vascular complex, a superficial capillary plexus, an intermediate capillary plexus, a deep capillary plexus, an outer retina plexus, a choriocapillaris plexus, an inner plexiform layer, an outer plexiform layer, and/or a choroid plexus. Each slab or plexus may span one or more layers of the retina or portions thereof.

The boundaries of the slab used may be defined by the location of the OCT scans that form the OCTA volume. Because OCT and OCTA scans acquired at a same tissue location are perfectly registered to each other, the OCT layers forming the OCTA volume may be segmented due to the contrast that exists in the OCTA image. An upper boundary and a lower boundary may be defined from these OCT layers or offsets of OCT layers, and a vascular flow signal value at every lateral location may be assigned through either averaging the vascular flow signal of the voxels contained between the boundaries, or finding the vascular flow signal of the voxel with the highest vascular flow within the boundaries.

For example, slab input 120 may include an inner retinal slab that is identified as the portion of the layers of OCTA volume 114 located between (inclusive or exclusive of) the inner limiting membrane (IML) and the outer boundary of the outer plexiform layer (OPL). This inner retinal slab may be a cross-sectional portion of OCTA volume 114 (e.g., with respect to an axial axis that, for example, extends in the inner to outer direction with respect to retinal anatomy). The inner retinal slab is the portion of OCTA volume 114 that includes the foveal avascular zone and allows for quantification of parameters relating to the foveal avascular zone. In other embodiments, slab input 120 may be identified as a sub-portion of the inner retinal slab. For example, slab input 120 may be a thinner portion of the inner retinal slab. However, in one or more embodiments, an upper boundary of the slab may always be selected as the inner limiting membrane to avoid challenges caused by the projection artifacts overlapping with in situ vascular flow signal, while the lower boundary definition may be varied. In other embodiments, both the upper boundary and lower boundary may be varied.

The boundaries of these slabs may be defined by the location of the OCT scans that form the OCTA volume. Because OCT and OCTA scans acquired at a same tissue location are perfectly registered to each other, the OCT layers forming the OCTA volume may be segmented due to the contrast that exists in the OCTA image. An upper boundary and a lower boundary may be defined from these OCT layers or offsets of OCT layers, and a vascular flow signal value at every lateral location may be assigned through either averaging the vascular flow signal of the voxels contained between the boundaries, or finding the vascular flow signal of the voxel with the highest vascular flow within the boundaries. While the upper boundary is always defined as corresponding to the inner limiting membrane (to avoid challenges caused by the projection artifacts overlapping with in situ vascular flow signal), the lower boundary definition may be varied.

FAZ analysis system 122 may include a model system 124. Model system 124 may be used to perform for image segmentation of slab input 120. For example, model system 124 may be used generate mask (or masked) images in which one or more features are segmented out. For example, a segmented image may be one in which a mask or some other graphical indicator is used to identify a selected feature(s) in an image. As one example, generating a mask image from an image input may include modifying the image input such that pixel values are designated either as being background (e.g., value “0”) or as being the selected features(s) (e.g., value of “1”).

In one or more embodiments, model system 124 includes one or more machine learning models. The one or more machine learning models may include, for example, one or more deep learning models. Further, the one or more deep learning models may include, for example, one or more neural networks including, but not limited to, convolutional neural networks (CNNs). For example, model system 124 may include one or more UNets, one or more convolutional layers, oner or more other types of layers or functions (e.g., pooling layers, sigmoid activation function, etc.), or a combination thereof.

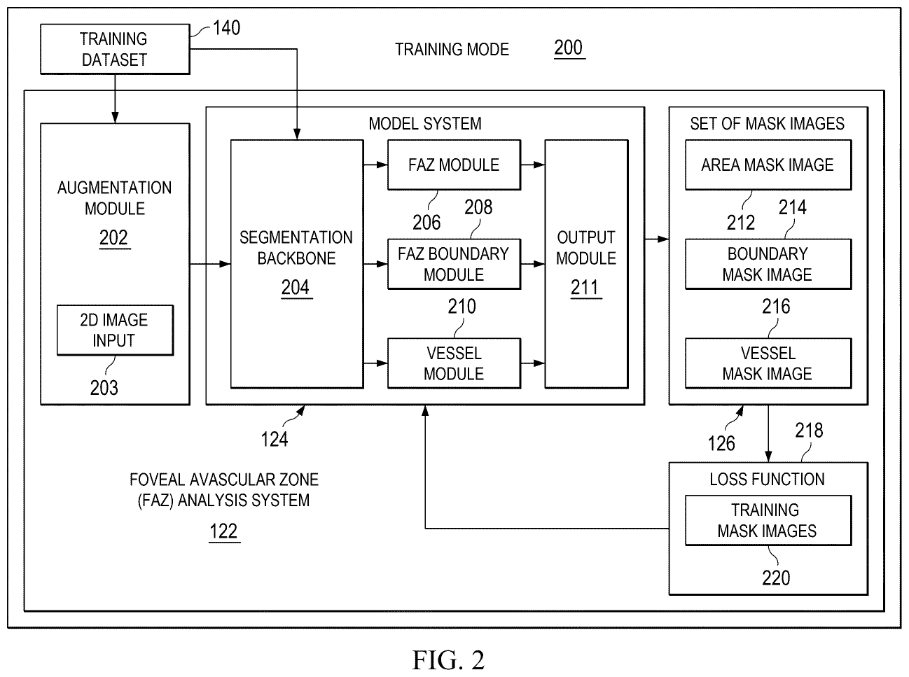

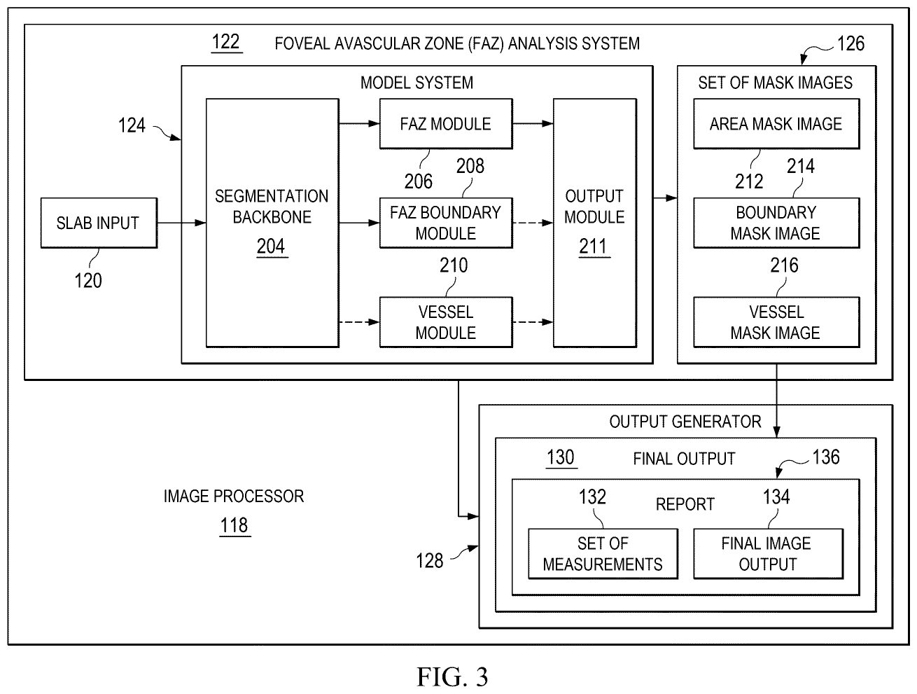

In one or more embodiments, model system 124 receives slab input 120 for processing and generates segmentation output 125. Segmentation output 125 identifies various features associated with the foveal vascular zone captured in the OCT imaging data 110. Segmentation output 125 includes set of mask images 126, which includes one or more mask images that relate to the foveal avascular zone (FAZ). For example, each mask image of set of mask images 126 may identify one or more selected features (e.g., a FAZ area, FAZ boundary, vessels) relating to the FAZ. One example of an implementation for model system 124 is described in further detail below with respect to FIGS. 2-3.

Image processor 108 may further include output generator 128. Output generator 128 may receive set of mask images 126 for processing to form final output 130. Final output 130 may take various forms.

For example, output generator 128 may process segmentation output 125 to generate set of measurements 132. Set of measurements 132 may include, but are not limited to, at least one of a first measurement for the area of the foveal avascular zone, a second measurement indicating a circularity of the foveal avascular zone, a third measurement indicating a tortuosity of the foveal avascular zone, vessel density, perfusion density, fractal dimension, vessel tortuosity index, vessel caliber index, or one or more other measurements.

In some embodiments, final output 130 may include final image output 131. Final image output 134 may include segmentation output 125 (e.g., all of segmentation output 125 or at least a portion of segmentation output 125), OCT imaging data 110 (e.g., all of OCT imaging data 110 or at least a portion of OCT imaging data 110), or both. For example, final output 130 may include set of mask images 126 or at least one mask image of set of mask images 126.

In some embodiments, final image output 134 includes a modified form of set of mask images 126 and/or a modified form of OCT imaging data 110. For example, output generator 128 may perform one or more operations on set of mask images 126 and/or OCT imaging data 110 to generate final image output 134. These operations may include, for example, at least one of scaling, cropping, resizing, flipping (horizontally, vertically, or both), rotating, changing pixel values of the mask and/or background of, adding annotations to, adding graphical indicators (e.g., labels, color, text, highlighting, etc.) to, reducing the noise of, or otherwise modifying set of mask images 126 and/or OCT imaging data 110.

In one or more embodiments, output generator 128 may generate final output 130 in the form of a report 136 that includes any one or more of the above-identified outputs and/or other information. For example, report 136 may include final image output 131, set of measurements 132, a combined form of both (e.g., final image output 131 that has been annotated to identify set of measurements 132), one or more other types of information, or a combination thereof.

For example, in one or more embodiments, report 136 may include an indication (or prediction) of a prognosis for the subject with respect to a retinal disease. The indication may include, for example, without limitation, a prediction of disease progression, such as, but not limited to, a predicted disease growth rate, a predicted future measured area for an area of the retina affected by the retinal diseases, a prediction of treatment response, and/or a prediction of disease burden.

In one or more embodiments, FAZ analysis system 122 may be trained in a training mode based on training dataset 140 to perform image segmentation and then used in an inference mode to generate segmentation output 125 based on slab input 120. One example of a method for training FAZ analysis system 122, including model system 124 of FAZ analysis system 122, is described in further detail in Section III below.

In one or more embodiments, analysis system 101 stores OCTA imaging data 110 obtained from imaging system 108 or a portion thereof, slab input 120 or a portion thereof, segmentation output 125 or a portion thereof, final output 130 or a portion thereof, other data generated during the processing of OCTA imaging data 110, or a combination thereof in data storage 104. In some embodiments, the portion of data storage 104 storing such information may be configured to comply with the security requirements of the Health Insurance Portability and Accountability (HIPAA) that mandate certain security procedures when handling patient data (e.g., such as OCT images of tissues of patients) (i.e., the data storage 104 may be HIPAA-compliant). For instance, the information being stored may be encrypted and anonymized. For example, the OCTA volume 110 may be encrypted as well as processed to remove and/or obfuscate personally identifying information of the subjects from which the OCTA volume 110 was obtained. In some instances, the communications link between imaging system 108 and analysis system 101 that utilizes network 116 may also be HIPAA-compliant. For example, at least a portion of network 122 may be a virtual private network (VPN) that is end-to-end encrypted and configured to anonymize personally identifying information data transmitted therein.

Image processing system 100 may be implemented using any number or combination of servers and/or software components that operate to perform various processes related to the capturing and processing of OCTA volumes of retinas. Examples of servers may include, for example, stand-alone and enterprise-class servers. In one or more embodiments, image processing system 100 may be operated and/or maintained by one or more different entities.

In some embodiments, OCTA imaging system 110 may be maintained by an entity that is tasked with obtaining OCTA imaging data 110 for tissue samples of subjects for the purposes of disease screening, diagnosis, disease monitoring, disease treatment, research, clinical trial management, or a combination thereof. For example, the entity may be a health care provider (e.g., ophthalmology healthcare provider) that seeks to obtain OCTA imaging data 110 for retinas of subjects for use in diagnosing retinal diseases and/or other types of eye conditions. As another example, the entity may be an administrator of a clinical trial that is tasked with collecting OCTA imaging data 110 for retinas of subjects to monitor retinal changes over the course of a disease, monitor treatment response, or both. Analysis system 101 may be maintained by a same or different entity (or entities) as imaging system 108. For example, analysis system 101 may be maintained by an entity that is tasked with identifying or discovering biomarkers of retinal diseases from OCT images.

### II.A. Example of FAZ Analysis System in Training Mode

FIG. 2 is a block diagram of the foveal avascular zone (FAZ) analysis system 122 from FIG. 1 described in further detail with respect to a training mode in accordance with one or more embodiments. As previously discussed, model system 124 of foveal avascular zone (FAZ) analysis system 122 may be trained using training dataset 140.

In one or more embodiments, training dataset 140 includes a plurality of training OCTA volumes. Training dataset 140 may include various types of OCTA volumes. For example, the plurality of training OCTA volumes may have been generated by a same OCTA imaging system (e.g., imaging system 108 in FIG. 1), different OCTA imaging systems that are of the same type, or two or more different types of OCTA imaging systems. In some cases, the raining dataset 140 may OCTA volumes of different quality even where produced by a same OCTA imaging system (or scanner).

The plurality of training OCTA volumes may include OCTA volumes for retinas associated with a same type of retinal disease (e.g., diabetic retinopathy), retinas associated with different stages of a same retinal disease or different types and/or stages of different retinal diseases, or a combination thereof. For example, the training dataset 140 may include different OCTA volumes of retinas exhibits different degrees of disease severity and/or different degrees of disease burden. Training dataset 140 may include OCTA volumes for retinas with different disease prognoses. The plurality of training OCTA volumes may include only one OCTA volume for the retina of a given subject, multiple OCTA volumes for the same retina of a given subject, multiple OCTA volumes for both retinas of a given subject, or a combination thereof. In this manner, the plurality of training OCTA volumes may be implemented in different ways.

In one or more embodiments, the training dataset 140 may include OCTA volumes for retinas all diagnosed with a particular retinal disease (e.g., nAMD, diabetic retinopathy, diabetic macular edema, etc.). In some embodiments, the training dataset 140 includes OCTA volumes that are partitioned into training OCTA volumes, validation OCT volumes, and test OCTA volumes. In some embodiments, the OCTA volumes of the training dataset 140 are partitioned into only training OCTA volumes and validation OCTA volumes.

In one or more embodiments, FAZ analysis system 122 includes augmentation module 202. Augmentation module 202 may be considered separate from or part of model system 124, depending on the implementation.

Augmentation module 202 may receive training dataset 140 and process training dataset 140 to form 2D image input 203 for training model system 124. 2D image input 203 may include a plurality of 2D projections for a corresponding plurality of OCTA slabs. One example of an implementation for forming 2D image input 203 is described in greater detail in Section III.

Model system 124 may include segmentation backbone 204, a plurality of modules (e.g., foveal avascular zone (FAZ) module 206, foveal avascular zone (FAZ) boundary module 208, and/or vessel module 210), and output module 211. In one or more embodiments, model system 124 may additionally include one or more other types of layers, modules, and/or algorithms.

In one or more embodiments, segmentation backbone 204 is the primary module for processing and segmenting 2D image input 203 for training model system 124. Segmentation backbone 204 may be implemented using one or more neural networks including, for example, without limitation, one or more convolutional neural networks. For example, segmentation backbone 204 may include the structure of at least one UNet (or U-Net) model. Segmentation backbone 204 may include, for example, the structure of a UNet model, the structure including, for example, contracting and expanding paths that are configured to then ultimately produce one or more feature maps. Accordingly, segmentation backbone 204 may also be referred to as a feature extraction module. Each feature map may include a set of key features representative of a corresponding 2D image (e.g., 2D projection of an OCTA slab).

The output of segmentation backbone 204 is fed as input into the plurality of modules. The plurality of modules may include at least two modules from FAZ module 206, FAZ boundary module 208, and vessel module 210. For example, the plurality of modules may include FAZ module 206 as well as boundary module 208 and/or vessel module 210. In one or more embodiments, each of these modules is implemented using one or more convolution layers. For example, each of FAZ module 206, FAZ boundary module 208, and vessel module 210 may be implemented using a single 1×1 convolutional layer that is used to compress the features of the feature map received as input. These layers may effectively function as the last or final layer of the UNet model. Thus, in some embodiments, these modules and the segmentation backbone 204 may be together considered all part of an overall UNet model.

The outputs of the plurality of modules are fed into output module 211. Output module 211 may be implemented using, for example, without limitation, a sigmoid activation function. Applying the sigmoid activation function to each output of the plurality of modules produces a corresponding mask images in set of mask images 126. For example, output module 211 may produce area mask image 212 based on the output of FAZ module 206; output module 211 may produce boundary mask image 214 based on the output of FAZ boundary module 208; and output module 211 may produce vessel mask image 216 based on the output of vessel module 210.

Area mask image 212 detects or identifies those pixels that represent the area that makes up the foveal avascular zone and distinguishes those pixels from background. For example, a “mask” is used to identify those pixels in which FAZ pixels have a first value and all other pixels have a second value. In some cases, the mask in area mask image 212 may be a highlighting of, coloring of, or some other type of graphical indication overlaid on a representation of the original 2D image that indicates that a group of pixels represents or belongs to the foveal avascular zone.

Boundary mask image 214 detects or identifies those pixels that represent the boundary of the foveal avascular zone and distinguishes those pixels from background. For example, a “mask” is used to identify those pixels in which FAZ boundary pixels have a first value and all other pixels have a second value. In some cases, the mask in boundary mask image 212 may be a highlighting of, coloring of, or some other type of graphical indication overlaid on a representation of the original 2D image that indicates that a group of pixels represents or belongs to the foveal avascular zone boundary.

Vessel mask image 216 detects or identifies those pixels that represent the vessels associated with the foveal avascular zone and distinguishes those pixels from background. The vessels associated with the foveal avascular zone are those vessels outside the foveal avascular zone. For example, a “mask” is used to identify those pixels in which vessel pixels have a first value and all other pixels have a second value. In some cases, the mask in boundary mask image 212 may be a highlighting of, coloring of, or some other type of graphical indication overlaid on a representation of the original 2D image that indicates the pixels that represent or belong to vessels associated with the foveal avascular zone.

Training model system 124 includes evaluating the loss for each epoch of training using loss function 218. Loss may be computed by loss function 218 for each epoch, with each epoch including batch processing with any number of batches per epoch based on 2D image input 203.

Loss function 218 may itself include one or more loss functions. In one or more embodiments, loss function 218 includes a cross entropy computed for each module and its corresponding output (mask image of the set of mask images) based on a plurality of training mask images 220, which function as ground truth for training purposes. The plurality of training mask images 220 include training area mask images, training boundary mask images, and training vessel mask images.

The cross entropy computed for FAZ module 206 quantifies a difference between area mask image 212 and the corresponding training area mask image. The cross entropy computed for FAZ boundary module 208 quantifies a difference between boundary mask image 214 and the corresponding training boundary mask image. The cross entropy computed for FAZ vessel module 210 quantifies a difference between vessel mask image 216 and the corresponding training vessel mask image.

Further, loss function 218 may take into account a weighted hausdorff distance, which is a distance-based metric that is applied to FAZ boundary module 208 to negatively affect (e.g., punish) those segmentations that are more circular and do not accurately capture the actual delineation of the foveal avascular zone. Using this type of distance metric for loss may increase a sharpness of the foveal avascular zone boundary identified by FAZ boundary module 208. Accurately capturing the FAZ boundary may be important because FAZ tortuosity may be a useful parameter in the prediction of DR or in indicating a prognosis for DR.

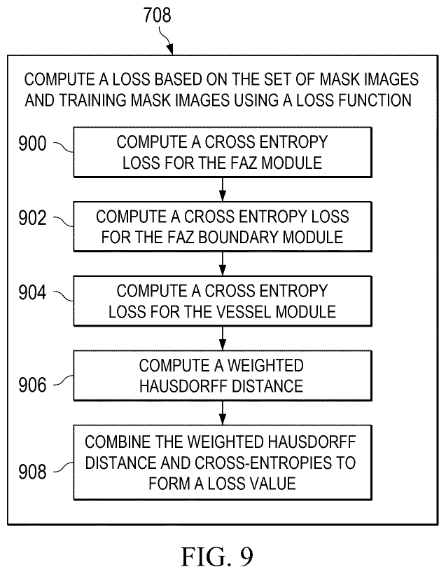

Loss function 218 may include various functions or types of losses. For example, loss function 218 may include cross-entropy loss, distribution-based loss, a region-based loss, a boundary based loss, a compounded loss, and/or one or more other types of loss. One example of an implementation for computing loss is described with respect to FIG. 9 below.

The plurality of training mask images 220 may be generated in a number of different ways. In one or more embodiments, the training area mask images are manually created by human graders. The training boundary mask images may be generated based on these training area mask images. For example, for a particular training area mask image, the corresponding boundary mask image may be generated based on the boundary of the area of the FAZ identified in that particular training area mask image. In some cases, edge detection may be used to generate the training boundary mask image.

Further, the training vessel mask images may be generated in different ways. In one or more embodiments, an amplitude threshold may be applied to a training area mask image. For example, an amplitude threshold may be applied to a 2D projection of an OCTA slab in order to remove the background and leave only pixels corresponding to vessels. This amplitude threshold may apply yielded extrafoveal vessel density and perfusion density distributions centered that are within tolerances of expected distributions. Amplitude thresholding may help ensure that vessel density is not impacted by a scan brightness such that better quality images would artificially show greater vessel density simply due to increased brightness.

After amplitude threshold, a machine learning algorithm (e.g., a clustering machine learning algorithm) may be used to identify the vessel mask image. This process may not be supervised and may occur in a semi-automated way in which the training vessel mask area that is generated is iteratively refined based on input form a human grader. In one or more embodiments, the clustering machine learning algorithm may take the form of a Density-Based Spatial Clustering Application with Noise (DBSCAN) algorithm, which is an unsupervised machine learning algorithm.

For each new epoch in the training process, augmentation module 202 may augment 2D image input 203. In other words, augmentation module 202 may modify 2D image input 203. Examples of different ways in which 2D image input 203 can be augmented are described in further detail in Section III. By augmenting 2D image input 203 for each epoch, a more robust training of model system 124 may be performed.

### II.B. Example of FAZ Analysis System in Inference Mode

FIG. 3 is a block diagram of the foveal avascular zone (FAZ) analysis system 122 from FIGS. 1-2 described in further detail in accordance with one or more embodiments. After model system 124 has been trained (e.g., as described with respect to FIG. 2), FAZ analysis system 122 may be used to perform FAZ segmentation in, for example, an inference mode. For example, FAZ analysis system 122 may be used to receive OCT imaging data 110 from FIG. 1, form slab input 120 based on OCT imaging data 110, and generate set of mask images 126.

In some embodiments, after training, model system 124 is only used to generate area mask image 212. In other embodiments, model system 124 is used to generate two or more area mask image 212, boundary mask image 214, and vessel mask image 216. Training model system 124 to perform segmentation of FAZ boundaries, vessels, or both in addition to FAZ area (e.g., as described with respect to FIG. 2) improves the performance of model system 124 with respect to generating area mask image 212. This type of training technique improves the accuracy of the area of the foveal avascular zone that is delineated.

As previously discussed, output generator 128 may process at least one mask image of set of mask images 126 to generate set of measurements 132. Set of measurements 132 may include, but is not limited to, at least one of a first measurement for the area of the foveal avascular zone, a second measurement indicating a circularity of the foveal avascular zone, a third measurement indicating a tortuosity of the foveal avascular zone, vessel density, perfusion density, vessel tortuosity index, fractal dimension, vessel caliber index, or one or more.

The first measurement for the area of the foveal avascular zone, the second measurement indicating a circularity of the foveal avascular zone, and the third measurement indicating a tortuosity of the foveal avascular zone may be computed based on area mask image 212. In some cases, the second measurement indicating a circularity of the foveal avascular zone, and the third measurement indicating a tortuosity of the foveal avascular zone may be computed using boundary mask image 212.

For example, the first measurement for the area of the foveal avascular zone may be computed as the sum of pixel areas within the mask of pixels identified by model system 124 as being the FAZ area in area mask image 212. Generating the second measurement for the circularity of the foveal avascular zone may include, for example, applying a filter to generate a fixed width (e.g., one-pixel wide) FAZ boundary around the area of the foveal avascular zone in area mask image 212. Further, the ratio between this boundary and the perimeter of a perfect circle corresponding to the same area is computed. The perfect circle may, for example, fully encompass the area of the foveal avascular zone or may be otherwise parallel to a circle that fully encompasses the area of the foveal avascular zone. The closer that ratio is to 1, the higher the circularity of the foveal avascular zone.

Tortuosity, in the context of the foveal avascular zone, may refer to the presence (or absence) of prominent bending angles (e.g., sharp contour changes) in the perimeter of the foveal avascular zone. Accordingly, in some cases, the tortuosity of the foveal avascular zone may be measured based on an average ratio of arch length to chord length for each pair of points along the perimeter of the foveal avascular zone. In some cases, to reduce computational burden, the tortuosity of the foveal avascular zone may correspond to the average ratio of arch length to chord length for every pair of inflection points along the perimeter of the foveal avascular zone. Moreover, in some cases, the tortuosity of the foveal avascular zone may be measured in two- and/or three-dimensions. For example, boundary mask image 214 or the boundary computed as described above based on area mask image 212 may be overlaid onto the original three-dimensional optical coherence tomography angiography (OCTA) slab input (or OCTA volume) in order to compute the tortuosity of the foveal avascular zone in a three-dimensional space.

One or more FAZ measurements may be computed based on vessel mask image 216 and/or the 2D image used to produce vessel mask image 216. For example, vessel density, perfusion density, tortuosity index, fractal dimension, and vessel caliber index may be computed using vessel mask image 216. In some cases, vessel density may be computed by applying a skeletonization algorithm to obtain a so-called “skeleton” representation of the vessels present in vessel mask image 216 (and thereby, the corresponding 2D image) in which each vessel has the same width (e.g., one pixel wide). Vessel perfusion density may correspond to the ratio between the quantity of pixels depicting vessels and the total quantity of pixels within an effective area of vessel mask image 216 (and thereby, the corresponding 2D image). This effective area for both vessel density and vessel perfusion density may be defined as the area outside of a circle centered at the fovea, the area outside of the vascular foveal avascular zone, the area outside of the structural foveal avascular zone, the area of the parafoveal, the area of the perifoveal, the area of the para foveal avascular zone, or the entire field of view.

In some cases, vessel tortuosity may refer to the presence (or absence) of large (or sharp) bending angles in the vessels. As such, the vessel tortuosity index associated with the plurality of vessels present in vessel mask image 216 (and thereby, the corresponding 2D image) may correspond to the average of the ratios between the distances of every connected pair of points following a vessel and the distances of every connected pair of points following a straight line. The fractal dimension of the plurality of vessels present in vessel mask image 216 (and thereby, the corresponding 2D image) may be determined by applying fractal box counting analysis. The vessel caliber index may correspond to the ratio between a first quantity of pixels in the mask of pixels identified by model system 124 as being a part of the microvascular structure and a second quantity of pixels in the corresponding vessel skeleton mask. The effective area associated with the vessel caliber index may be defined to increase the sensitivity of this measurement to a particular disease. For instance, the large vessels may be excluded such that only capillaries contribute to the vessel caliber index. Alternatively, and/or additionally, dilated vessels may be segmented separately, and the mask of dilated vessels could be used as the effective area in order to exclude dilated vessels from the vessel caliber index.

### II.C. Example OCTA Slab Input

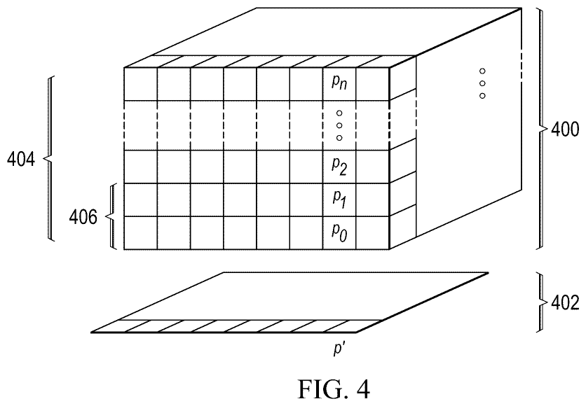

FIG. 4 is a schematic diagram illustrating the creation of a two-dimensional image from an OCTA volume in accordance with one or more embodiments. OCTA volume 400 may be processed to generate two-dimensional (2D) image 402, which may be one example of a 2D image that may be present in 2D image input 203 described with respect to FIG. 2. OCTA volume 400 may be one example of an implementation for OCTA volume 112 in FIG. 1.

Further, 2D image 400 is one example of augmentation that may be performed. For example, from OCTA volume 112, augmentation module 202 from FIG. 2 may identify a particular slab 404 (e.g., portion of the layers that make up OCTA volume 112. This slab 404 may be, for example, an internal retinal slab that is defined as the portion of OCTA volume 112 between the inner limiting membrane (ILM) and the outer plexiform layer (OPL).

Augmentation module 202 from FIG. 2 may then generate 2D image 402 as a 2D projection of slab 404. This projection may be done in different ways such that a pixel value in 2D image 402 is based on the one or more of the various intensity values of the corresponding stack or column of pixels (e.g., having a same x,y location) in slab 404. For example, for each column of pixels in slab 404, a mean intensity value for those pixels may be used as the pixel value for the corresponding pixel in 2D image 402. Alternatively, for each column of pixels in slab 404, a maximum intensity value for those pixels may be used as the pixel value for the corresponding pixel in 2D image 402.

As one example, the intensity value for the pixel p′ in 2D image 402 may be determined based at least on an intensity value of each of the pixels p₀, p₁, p₂, . . . , pₙ in the OCTA volume 400. For instance, in some cases, the intensity value of the pixel p′ in 2D image 402 may correspond to a maximum intensity value, an average intensity value, a minimum intensity value, a sum intensity value, a median intensity value, or a standard deviation intensity value of the set of intensity values associated with the stack of adjacent pixels p₀, p₁, p₂, . . . , pₙ in the 2D image 402.

In other embodiments, OCTA volume 400 may be used to identify a different slab 406 for the projection that forms 2D image 402 or, alternatively, slab 404 may be further augmented at a future epoch to form the projection that forms 2D image 402. For example, slab 406 may be a “thinner” version of slab 404. 2D image 402 may be created from slab 406 in a manner similar to the above described manner used to create 2D image 402 from slab 404.

### II.D. Example Mask Images in an Example Workflow

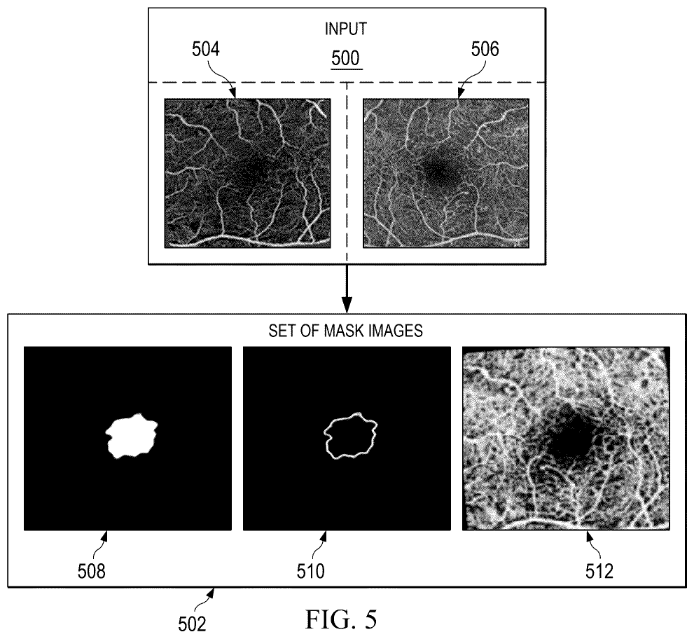

FIG. 5 is a schematic diagram of a workflow for generating a set of mask images in accordance with one or more embodiments. In the workflow shown in FIG. 5, input 500 may be processed by FAZ analysis system 122 described with respect to FIGS. 1-3 to form set of mask images 502.

Input 500 may be one example of an implementation for a slab included in slab input 120 in FIGS. 1 and 3 or a 2D image included in 2D image input 203 in FIG. 2. For example, when FAZ analysis system 122 in FIG. 1 is in a training mode, input 500 may take the form of 2D image 504. 2D image 504 may be a projection (e.g., such as 2D image 402 in FIG. 4) of a slab formed based on an internal retinal slab. The slab may be the internal retinal slab or some sub-portion of the internal retinal slab. When FAZ analysis system 122 in FIG. 1 is in an inference mode, input 500 may take the form of slab 506. Slab 560 may be an internal retinal slab or some sub-portion of the internal retinal slab.

Set of mask images 502 include area mask image 508, boundary mask image 510, and vessel mask image 512. Area mask image 508, boundary mask image 510, and vessel mask image 512 are examples of implementations for area mask image 212, boundary mask image 214, and vessel mask image 216, respectively.

### III. Example Methodologies for FAZ Area Segmentation

### III.A. Generating Mask Images and Retinal Disease Outputs

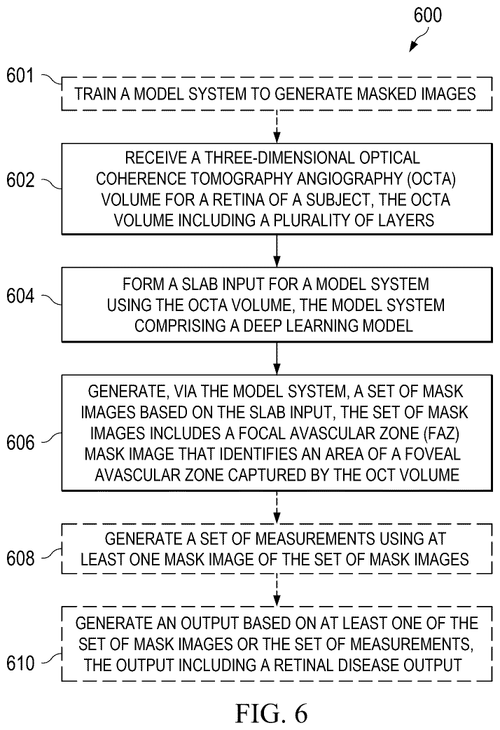

FIG. 6 is a flowchart of a process for analyzing an OCTA volume of a retina of a subject in accordance with one or more example embodiments. Process 600 in FIG. 6 may be implemented using analysis system 101 in FIGS. 1-3. In one or more embodiments, at least some of the steps of the process 600 may be performed by the processors of a computer or a server implemented as part of analysis system 101. It is understood that additional steps may be performed before, during, or after the steps of process 600 discussed below. In addition, in some embodiments, one or more of the steps may also be omitted or performed in different orders.

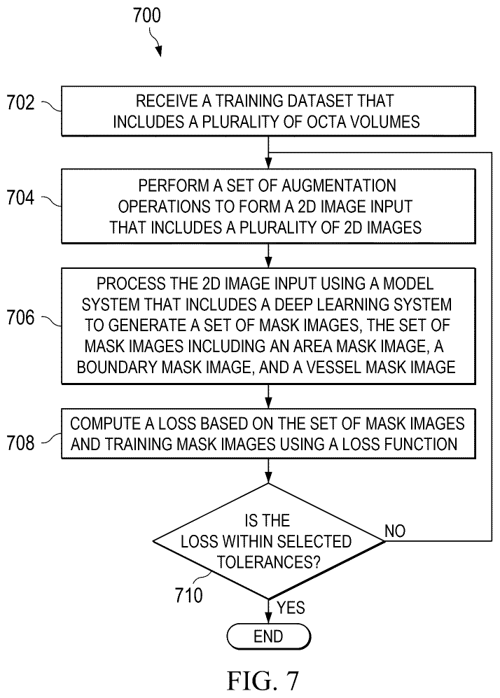

Process 600 may optionally include the step 601 of training a model system to generate masked images. One example of an implementation for step 601 is described below with respect to FIG. 7. The model system may be, for example, model system 124 in FIGS. 1-3. The model system may include a deep learning model. The model system may include, for example, one or more convolutional neural networks (CNN).

Step 602 of process 600 includes receiving a three-dimensional optical coherence tomography angiography (OCTA) volume for a retina of a subject, the OCTA volume including a plurality of layers. The plurality of layers may correspond with, for example, a plurality of optical coherence tomography (OCT) scans of the retina acquired at a same tissue location. The OCTA volume may be, for example without limitation, OCTA volume 110 in FIG. 1 or OCTA volume 400 in FIG. 4. The OCTA volume may capture the retina of a subject that is healthy or associated with retinal disease. The retinal disease may be, for example, age-related macular degeneration (AMD), neovascular age-related macular degeneration (nAMD), diabetic retinopathy (DR), macular edema, geographic atrophy, or some other type of retinal disease.

Step 604 of process 600 includes forming a slab input for a model system using the OCTA volume, the model system comprising a deep learning model. The slab input may be, for example, slab input 120 in FIG. 1. The slab input may be, for example, an internal retinal slab of the OCTA volume that includes the portion of the OCTA volume between the ILM and OPL. In some cases, the slab input is a sub-portion of this internal retinal slab. In some embodiments, the slab input is formed by identifying the internal retinal slab or a portion thereof as the selected slab and then preprocessing the selected slab to form the slab input. In some cases, the preprocessing is performed on the OCTA volume prior to identifying the slab. The preprocessing may include, for example, performing a set of preprocessing operations that includes at least one of a normalization operation, a scaling operation, a resizing operation, a horizontal flipping operation, a vertical flipping operation, a cropping operation, a rotation operation, a noise filtering operation, or some other type of preprocessing operation.

Step 606 of process 600 includes generating, via the model system, a set of mask images based on the slab input, the set of mask images includes a focal avascular zone (FAZ) mask image that identifies an area of a foveal avascular zone captured by the OCT volume. The set of FAZ mask images may be, for example without limitation, set of mask images 126 in FIGS. 1-3 or set of mask images 502 in FIG. 5. In addition to the area mask image, the set of mask images may include a boundary mask image that identifies a boundary of the foveal avascular zone and/or a vessel mask image that identifies vessels associated with the foveal avascular zone.

Process 600 may optionally include step 608, which includes, generating a set of measurements using at least one mask image of the set of mask images. The set of measurements may include, for example, at least one of a first measurement for the area of the foveal avascular zone, a second measurement indicating a circularity of the foveal avascular zone, a third measurement indicating a tortuosity of the foveal avascular zone, a vessel density, a perfusion density, a fractal dimension, a vessel tortuosity index, or a vessel caliber index. The set of measurements may be, for example, set of measurements 132 described with respect to FIGS. 1 and 3.

Process 600 may optionally include step 610, which includes generating an output based on at least one of the set of mask images or the set of measurements, the output including a retinal disease output. The retinal disease output may include, for example, a prediction of a retinal disease developing, a prediction about retinal disease progression, an indication about a prognosis for the subject with respect to a retinal disease, or a combination thereof.

Process 600, which may be implemented using image processing system 100 described in FIG. 1 or at least analysis system 101 in FIG. 1, provides an improvement to the technical field of retinal disease screening, diagnosis, and treatment management. For example, by improving the accuracy, precision, and reliability of FAZ area segmentation, process 600 thereby improves the accuracy, precision, and reliability of quantitative measurements made based on FAZ area segmentation, improves the identification of biomarkers for retinal disease and/or treatment response, and improves segmentation of OCTA images. These improvements may be realized regardless of the type of OCTA imaging system used to generate the OCTA volume, the type of scanning protocol used to generate the OCTA volume, the quality of the OCTA volume, or a type of disrupting pathology or abnormality present in the retina. Accordingly, process 600 may facilitate improved automatic analysis of large datasets of OCTA volumes even in the presence of varying conditions associated with the OCTA volumes.

### III.B. Training a Model System to Generate Mask Images

FIG. 7 is a flowchart of a process for training a model system to generate a set of mask images in accordance with one or more embodiments. Process 700 in FIG. 7 may be implemented using analysis system 101 in FIGS. 1-3. Process 700 may be one example of an implementation for step 601 of process 600 in FIG. 6. Further, it is understood that additional steps may be performed before, during, or after the steps of process 700 discussed below. In addition, in some embodiments, one or more steps may also be omitted or performed in different orders.

Step 702 of process 700 includes receiving a training dataset that includes a plurality of optical coherence tomography angiography (OCTA) volumes for a plurality of retinas. The training dataset may be, for example, training dataset 140 described with respect in FIGS. 1-2. As described above, the training dataset used to train the model may include various types of OCTA volumes. The training dataset may include OCTA volumes for retinas of varying health conditions. In one or more embodiments, the training dataset may include one or more OCTA volumes for healthy retinas. In one or more embodiments, the training dataset may include OCTA volumes for retinas diagnosed with a retinal disease such as AMD, nAMD, diabetic retinopathy, macular edema, geographic atrophy, or some other type of retinal disease. In some cases, the training data may include one or more OCTA volumes for damaged retinas. The training dataset may include OCTA volumes for a same type of retina (e.g., healthy or diseased or damaged) or different types of retinas. In some instances, the plurality of OCTA volumes may be generated by more than one OCTA imaging system (or type of OCTA imaging system).

In some embodiments, the training dataset includes OCTA volumes that are partitioned into training OCTA volumes, validation OCT volumes, and test OCTA volumes. In some embodiments, the OCTA volumes of the training dataset are partitioned into only training OCTA volumes and validation OCTA volumes.

The training dataset may be formed in a manner that helps prevent or reduce the possibility of the model system learning spurious system-specific features. Accordingly, after training according to one or more of the embodiments described herein, the model system may be capable of performing segmentation in a system-agnostic manner (e.g., without being specific to a particular OCTA imaging system or scanner).

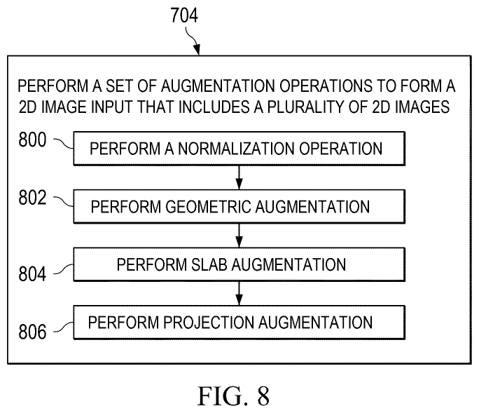

Step 704 of process 700 includes performing a set of augmentation operations to form a 2D image input that includes a plurality of 2D images. The 2D image input may be, for example, 2D image input 203 in FIG. 2. The set of augmentation operations may be performed by, for example, augmentation module 202 in FIG. 2. In one or more embodiments, step 704 may be performed in a way that increases the overall number of images that can be used to train the model system. For example, the set of augmentation operations may be used to turn one OCTA volume into multiple (e.g., 3, 4, 5, 6, 7, 8, 9, 10, 11, 15, 20, 30, 50, etc.) 2D images that can be used for training at different training cycles (e.g., epochs). One example of a manner in which step 704 may be performed is described with respect to FIG. 8 below.

Step 706 of process 700 includes processing the 2D image input using a model system that includes a deep learning system to generate a set of mask images, the set of mask images including an area mask image, a boundary mask image, and a vessel mask image. The model system may be, for example, model system 124 described with respect to FIGS. 1-3. The model system may include, for example, at least one convolutional neural network (e.g., a UNet).

Step 708 of process 700 includes computing a loss based on the set of mask images and training mask images using a loss function.

Step 710 of process 700 includes determining whether the loss is within selected tolerances. In other words, a determination is made as to whether the loss has been sufficiently minimized.

If the loss is within tolerances, the model system is considered trained and ready for future use in an inference mode and the process 700 terminates. The model system may be used accurately and reliably generate the set of FAZ mask images. If, however, the loss is not within selected tolerances, the process 700 process to operation 704 as described above to begin a next cycle (e.g., epoch) of training. Thus, for each next training cycle, a new set of augmentation operations may be performed to form the 2D image input used for the next training cycle.

FIG. 8 is flowchart of a process for performing a set of augmentation operations in accordance with one or more example embodiments. FIG. 8 depicts one example of an implementation of a process for performing operation 704 described above with respect to FIG. 7. Additional steps may be performed before, during, or after the steps discussed below as part of the process of performing step 704. Further, in some embodiments, one or more steps may be omitted or one or more steps may be performed in different orders.

As previously discussed, step 704 includes performing a set of augmentation operations to form a 2D image input that includes a plurality of 2D images. At the initial training cycle (or epoch), the set of augmentation operations is performed with respect to the plurality of OCTA volumes received in the training dataset. In one or more embodiments, performing step 704 may include performing step 800, step 802, step 804, step 806, or a combination thereof. In one or more embodiments, step 704 always includes performing step 806 and may additionally include performing at least one of step 800, step 802, and step 804.

Operation 800 includes performing a normalization operation. The normalization operation may be performed to normalize the pixel values (e.g., intensity values) across the plurality of OCTA volumes. Normalization may be performed to normalize images produced by the same type of imaging system using the corresponding set of mean and standard deviation. Normalization may include subtracting by the mean and dividing by the standard deviation, which results in zero mean and unit standard deviation within the images produced by the same imaging system.

In other cases, system information may be ignored. For example, when the plurality of OCTA volumes includes OCTA volumes generated by different imaging systems or different types of imaging systems, the different OCTA volumes may use different ranges of intensity values, normalization can help ensure the model system is trained to perform segmentation in a manner that is system-agnostic (e.g., not specific to any particular type of imaging system, configuration settings, intensity value ranges, etc.). For example, all of plurality of OCTA volumes may be normalized with respect to a single set of mean and standard deviation regardless of imaging system. In some cases, the augmentation module includes a learnable normalization layer, which may or may not take into account imaging system information when normalizing the images prior to segmentation.

Operation 802 includes performing a geometric augmentation. Geometric augmentation may include, for example, without limitation, one or more operations that affect the geometric layout of plurality of OCTA volumes. For example, performing geometric augmentation may include at least one of performing a scaling operation, a resizing operation, a horizontal flipping operation, a vertical flipping operation, a cropping operation, a rotation operation, or some other type of operation.

Operation 804 includes performing a slab augmentation. Slab augmentation may include, for example, identifying a slab of each OCTA volume for use in training. The slab is a 3D portion of the OCTA volume. In one or more embodiments, slab augmentation includes identifying the internal retinal slab of each OCTA volume for use in training. In some embodiments, the internal retinal slab may already be identified as part of training dataset. The internal retinal slab portion may be the portion of the OCTA volume between (inclusive or exclusive, depending on the implementation) between the IML and OPL of the retina. In one or more embodiments, slab augmentation includes identifying a sub-portion of the internal retinal slab for use in training (e.g., superficial vascular complex (SVC), inner plexiform layer (IPL)).

Using slabs that are, for example, thinner than those used in clinical applications (e.g., the internal retinal slab based on the nerve fiber layer boundary) helps simulate a high ischemic state since such that any projection (or 2D image) generated based on that slab would contain less blood flow between boundaries. In one or more embodiments, any slab augmentation still uses the inner limiting membrane as a boundary for the slab to help avoid challenges that may be caused by projection artifacts overlapping with in situ flow signal.

Operation 806 includes performing a projection augmentation. In one or more embodiments, projection augmentation includes converting a 3D image into 2D (e.g., converting a slab into a 2D image). These types of projections may be referred to as en face visualizations of the slab.

Performing projection augmentation may occur in different ways. For example, if the pixel in each layer of the slab is given an x,y location, the 2D image that is formed has the same size as the x,y plane of the slab. Each pixel in the 2D image (projection) is given a value that is based on the intensity values of the pixels in the stack (or column) of pixels in the slab at that x,y location. For example, of the pixels included in the slab in each layer of the slab for a given x,y location (e.g., with each layer being a cross-section with respect to the z-axis), the maximum intensity value, average (or mean) intensity value, a minimum intensity value, a sum intensity value, a median intensity value, or a standard deviation intensity value identified may be assigned to the corresponding pixel at the x,y location of the 2D image (projection). Operation 804 and operation 806 together allow differences in contrast, signal to noise ratio, and capillary density to help improve robustness of the training of the model system.

In this manner, performing step 704 as described herein may allow diversity in the data that is used to train the model system. This diversity may improve the robustness of the model system and improve overall model system performance. Further, introducing the type of diversity discussed herein may help avoid underfitting and overfitting the model system. Step 704 may include other types of augmentation operations in addition to the ones described above. For example, step 704 may include a noise filtering operation. Step 704 may include operations to remove motion artifacts. For example, step 704 may include operations to perform registration and/or merging to remove motion artifacts.

FIG. 9 is flowchart of a process for computing a loss based on a set of mask images and training mask images using a loss function in accordance with one or more example embodiments. FIG. 9 depicts one example of an implementation of a process for performing operation 708 described above with respect to FIG. 7. Additional steps may be performed before, during, or after the steps discussed below as part of the process of performing step 708. Further, in some embodiments, one or more steps may be omitted, or one or more steps may be performed in different orders.

As previously discussed, step 708 includes computing a loss based on the set of mask images generated in process 700 in FIG. 7 and training mask images using a loss function. The loss function may be, for example, loss function 218 described with respect to FIG. 2 above.

In one or more embodiments, performing step 708 may include performing step 900, step 902, step 904, step 906, 908, or a combination thereof. In one or more embodiments, step 704 always includes performing all of steps 902-908.

Step 900 includes computing a cross entropy loss for the FAZ module. The cross-entropy computed may quantify a difference between the area mask image generated by the FAZ module and the corresponding training area mask image (e.g., ground truth, which may be manually created by a human grader).

Step 902 includes computing a cross entropy loss for the FAZ boundary module. The cross-entropy computed may quantify a difference between the boundary mask image generated by the FAZ boundary module and the corresponding training boundary mask image (e.g., ground truth, which may be generated, for example, by a model or algorithm based on the training area mask image).

Step 904 includes computing a cross entropy loss for the vessel module. The cross-entropy computed may quantify a difference between the vessel mask image generated by the vessel module and the corresponding training vessel mask image. The training vessel mask image may be generated based on the training area mask image using, for example, amplitude threshold and a machine learning algorithm (e.g., a clustering ML algorithm such as, but not limited to, DBSCAN). Although steps 900, 902, and 904 describe computing cross-entropy loss, in other embodiments, some other type of loss metric may be computed in each of these steps.

Step 906 includes computing a weighted Hausdorff distance. The weighted Hausdorff distance is a distance-based metric that is applied to the FAZ boundary module to negatively impact (e.g., punish) those segmentations (boundary mask images) that are more circular than desired and do not capture the accurate delineation of the FAZ with the desired level of accuracy.

Step 908 includes combining the weighted Hausdorff distance and cross-entropies to form a loss value. For example, in one or more embodiments, the weighted Hausdorff distance and cross-entropies may simply be summed. In some embodiments, the cross-entropies may be averaged and summed with the weighted Hausdorff distance. In other examples, some other type of combining of these values may be performed to arrive at a total loss for the current training cycle of the training process.

Iv. Examples of Images Used in Training and Experimental Data

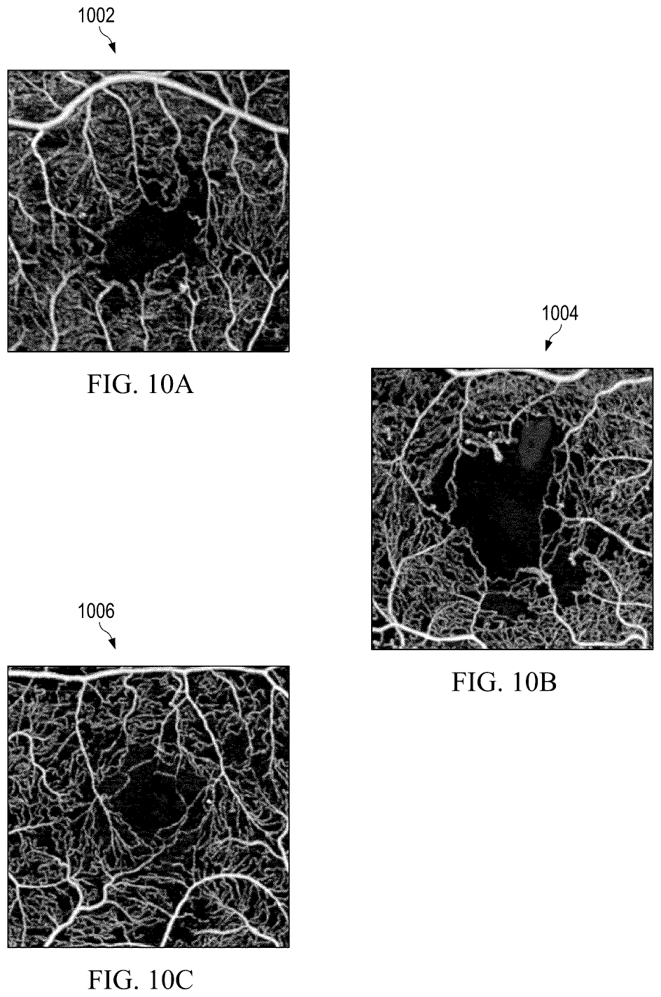

FIGS. 10A-10C depict examples of two-dimensional projections of slabs of three-dimensional optical coherence tomography angiography (OCTA) volumes exhibiting different disease observations associated with diabetic retinopathy in accordance with one or more embodiments. For example, in FIG. 5A, the retina in projection 1002 exhibits an irregular foveal avascular zone. In FIG. 5B, the retina in projection 1004 exhibits an enlarged foveal avascular zone, microaneurysms, and dilated vessels. In FIG. 5C, the retina in projection 1006 exhibits capillary loss (FIG. 5C). Projections 1002, 1004, and 1006 are examples of 2D images that may be included in, for example, 2D image input 203 in FIG. 2.

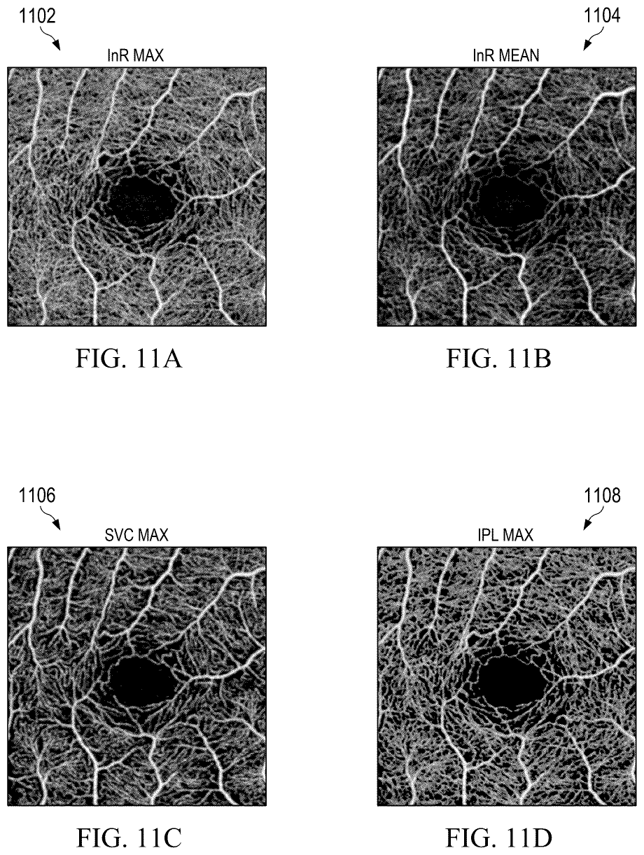

FIGS. 11A-11D depict examples of different types of augmentation that may be performed as part of training a model system (e.g., model system 124 in FIGS. 1-3) in accordance with one or more example embodiments. For example, a same OCTA slab such as, for example, an internal retinal slab (InR), may undergo various types of augmentation including, but not limited to, normalization, geometric augmentation, slab augmentation, and projection augmentation.

In FIG. 11A, image 1102 is a first projection of an internal retinal slab that has undergone at least projection augmentation in which the internal retinal slab has been converted to a 2D projection based on the maximum intensity value in each stack (or column) of pixels in the internal retinal slab, with the column running in the direction from IML to OPL.

In FIG. 11B, image 1104 is a second projection of the same internal retinal slab that has undergone a different form of projection augmentation in which the internal retinal slab has been converted to a 2D projection based on the mean intensity value for pixels in each stack (or column) of pixels in the internal retinal slab, with the column running in the direction from IML to OPL.

In FIG. 11C, image 1106 is a third projection of the same internal retinal slab that has undergone a slab augmentation and projection augmentation in which the internal retinal slab has been augmented to form a new slab that is a sub-portion of the internal retinal slab that includes only the superficial vascular complex (SVC). Further, this new slab undergoes projection augmentation in which the new slab is converted to a 2D projection based on the maximum intensity value for pixels in each stack (or column) of pixels in the OCTA slab, with the column running in the direction from IML to OPL.

In FIG. 11D, image 1108 is a fourth projection of the same internal retinal slab that has undergone yet another slab augmentation and projection augmentation in which the internal retinal slab has been augmented to form a new slab that is a sub-portion of the internal retinal slab that includes only the inner plexiform layer (IPL). Further, this new slab undergoes projection augmentation in which the new slab is converted to a 2D projection based on the maximum intensity value for pixels in each stack (or column) of pixels in the OCTA slab, with the column running in the direction from IML to OPL.

Thus, images 1102-1108, which are examples of images that may be included in 2D image input 203 in FIG. 2, illustrate how an OCTA slab (e.g., the internal retinal slab) may be augmented in various ways using slab augmentation and/or projection augmentation. In other embodiments, other types of augmentation may be performed including, for example, geometric augmentation (e.g., rotation, flipping, cropping, scaling, resizing, etc.), noise reduction, normalization, and/or a combination thereof.

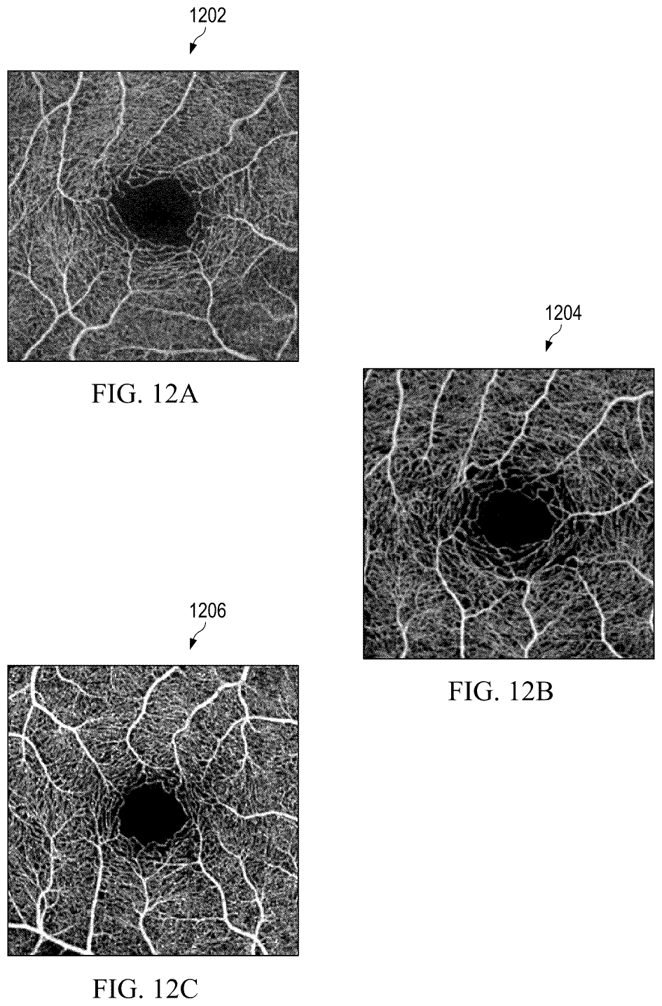

FIGS. 12A-12C depict examples of two-dimensional projections of slabs of OCTA volumes produced by three different imaging systems (e.g., OCTA scanners) in accordance with one or more embodiments. The configurations and settings of the different imaging systems may produce OCTA volumes that exhibit certain system-specific features which may skew the performance of the model system (e.g., model system 124 in FIGS. 1-3) if the model system is trained using an unbalanced training set that is formed based on a disproportionate quantity of OCTA volumes from one OCTA imaging system versus another OCTA imaging system.

In FIG. 12A, projection 1202 is acquired from a first OCTA imaging system.

In FIG. 12B, projection 1204 is acquired from a second OCTA imaging system.

In FIG. 12C, projection 1206 is acquired from a third OCTA imaging system.

The first, second, and third OCTA imaging systems may be different systems (e.g., different OCTA scanners). In one or more embodiments, these three OCTA imaging systems may all be of a same type of OCTA scanner but may operate with different configurations/settings. In some embodiments, at least two of the three OCTA imaging systems may be of different types.

Training the model system (e.g., model system 124 in FIGS. 1-3) using the projections from different OCTA imaging systems and/or types of OCTA imaging systems may allow the model system to be trained to perform in a system-agnostic manner. Accordingly, the performance of the model system may remain generally consistent across different OCTA imaging systems including, for example, swept-source scanner, spectral domain scanner, and/or the like.

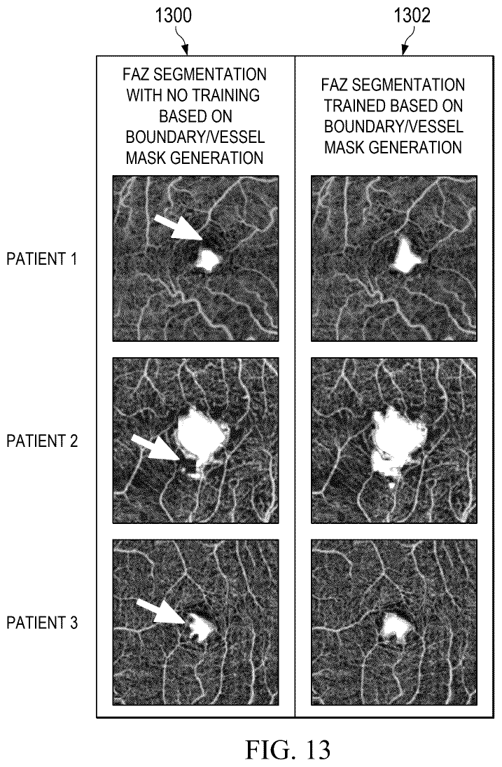

FIG. 13 depicts a comparison of training a model system in two different ways in accordance with one or more embodiments. In one or more embodiments, set of mask images 1300 is generated using a model system (e.g., model system 124 in FIGS. 1-3) that is trained to segment the area of the foveal avascular zone and excludes consideration of the boundary and vessels associated with the foveal avascular zone. In other words, for example, either the model system excludes the FAZ boundary module and the vessel module or the loss function used to train the model system does not take into account the outputs of the FAZ boundary module and the vessel module.

Set of mask images 1302 is generated using a model system (e.g., model system 124 in FIGS. 1-3) that is trained to segment the area of the foveal avascular zone and does take into consideration the boundary and vessels associated with the foveal avascular zone. In other words, for example, the model system includes the FAZ boundary module and vessel module and the loss function used to train the model system takes into account the outputs of the FAZ boundary module and the vessel module. Set of mask images 1302 more accurately capture the area of the foveal avascular zone than set of mask image 1300.

The arrows associated with set of mask images 1300 show undersegmentation of the FAZ area. These undersegmented areas are accurately captured in the set of mask images 1302.

### Experimental Data:

A model system implemented in a manner similar to model system 124 of FIGS. 1-3 was implemented. The model system was trained based on OCTA volumes generated from different scanners. Table 1 below indicates the performance of the trained model system across these different scanners, demonstrating the efficacy of using the trained model system in a system-agnostic manner:

TABLE 1

| | Scanner A | Scanner B | Scanner C |
|---|---|---|---|
| Validation Dice Score | 0.9177 | 0.9851 | 0.9635 |
| Test Dice Score | 0.8812 | 0.5868 | 0.9257 |

As shown above in Table 1, the performance of the trained model system on a validation set and a test set was assessed based on a Sørensen-Dice coefficient, which measures a similarity (or difference) between the segmentation of a foveal avascular zone, a foveal avascular zone boundary, and a plurality of vessels determined by the trained model system and the corresponding ground truth segmentations. A higher Sørensen-Dice coefficient indicates a greater match between the segmentation of the foveal avascular zone, the foveal avascular zone boundary, and the plurality of vessels determined by the trained model 135 and the corresponding ground truth segmentations.

Table 2 below shows that segmentation performance of the FAZ area is improved training the model system to identify the FAZ boundary and the plurality of vessels surrounding the FAZ. The error of measuring FAZ area and circularity both improved with this type of training:

TABLE 2

| | FAZ | Boundary | Vessel | Both |
|---|---|---|---|---|
| | Area | Task | Task | Tasks |
| | Alone | Added | Added | Added |
| Error of FAZ | 0.05 | 0.05 | 0.04 | 0.03 |
| area | | | | |
| Circularity | 0.08 | 0.09 | 0.05 | 0.05 |

### V. Example Computing System

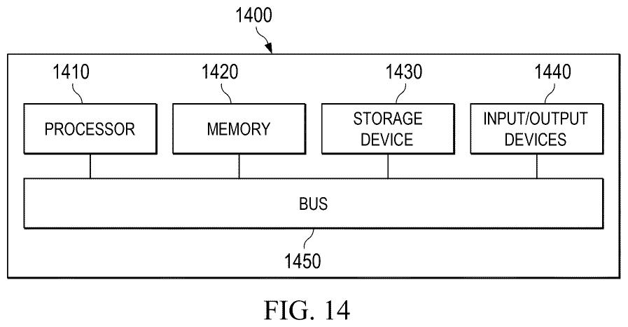

FIG. 14 is a block diagram illustrating an example of a computing system, in accordance with one or more example embodiments. Computing system 1400 may be used to implement computing platform 102 in FIG. 1 and/or any components therein.

As shown in FIG. 14, the computing system 1400 can include a processor 1410, a memory 1420, a storage device 1430, and input/output devices 1440. Computing system 1400 may be one example implementation of analysis system 101 in FIG. 1. The processor 1410, the memory 1420, the storage device 1430, and the input/output devices 1440 can be interconnected via a system bus 1450. The processor 1410 is capable of processing instructions for execution within the computing system 1400. Such executed instructions can implement one or more components of FIG. 1, for example, the analysis engine 101, and/or the like. In some example embodiments, the processor 1410 can be a single-threaded processor. Alternately, the processor 1410 can be a multi-threaded processor. The processor 1410 is capable of processing instructions stored in the memory 1420 and/or on the storage device 1430 to display graphical information for a user interface, such as display system 106 in FIG. 1.

The memory 1420 is a computer readable medium such as volatile or non-volatile that stores information within the computing system 1400. The memory 1420 can store data structures representing configuration object databases, for example. The storage device 1430 is capable of providing persistent storage for the computing system 1400. The storage device 1430 can be a floppy disk device, a hard disk device, an optical disk device, or a tape device, or other suitable persistent storage means. Storage device 1430 may be one example implementation of data storage 104 in FIG. 1 The input/output device 1440 provides input/output operations for the computing system 1400. In some example embodiments, the input/output device 1440 includes a keyboard and/or pointing device. In various implementations, the input/output device 1440 includes a display unit for displaying graphical user interfaces.

According to some example embodiments, the input/output device 1440 can provide input/output operations for a network device. For example, the input/output device 1440 can include Ethernet ports or other networking ports to communicate with one or more wired and/or wireless networks (e.g., a local area network (LAN), a wide area network (WAN), the Internet).

In some example embodiments, the computing system 1400 can be used to execute various interactive computer software applications that can be used for organization, analysis and/or storage of data in various formats. Alternatively, the computing system 1400 can be used to execute any type of software applications. These applications can be used to perform various functionalities, e.g., planning functionalities (e.g., generating, managing, editing of spreadsheet documents, word processing documents, and/or any other objects, etc.), computing functionalities, communications functionalities, etc. The applications can include various add-in functionalities or can be standalone computing products and/or functionalities. Upon activation within the applications, the functionalities can be used to generate the user interface provided via the input/output device 1440. The user interface can be generated and presented to a user by the computing system 1400 (e.g., on a computer screen monitor, etc.).

One or more aspects or features of the subject matter described herein can be realized in digital electronic circuitry, integrated circuitry, specially designed ASICs, field programmable gate arrays (FPGAs) computer hardware, firmware, software, and/or combinations thereof. These various aspects or features can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device. The programmable system or computing system may include clients and servers. A client and server are generally remote from each other and typically interact through a communication network. The relationship of client and server arises by virtue of computer programs running on the respective computers and having a client-server relationship to each other.

These computer programs, which can also be referred to as programs, software, software applications, applications, components, or code, include machine instructions for a programmable processor, and can be implemented in a high-level procedural and/or object-oriented programming language, and/or in assembly/machine language. As used herein, the term “machine-readable medium” refers to any computer program product, apparatus and/or device, such as for example magnetic discs, optical disks, memory, and Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor. The machine-readable medium can store such machine instructions non-transitorily, such as for example as would a non-transient solid-state memory or a magnetic hard drive or any equivalent storage medium. The machine-readable medium can alternatively or additionally store such machine instructions in a transient manner, such as for example, as would a processor cache or other random access memory associated with one or more physical processor cores.

To provide for interaction with a user, one or more aspects or features of the subject matter described herein can be implemented on a computer having a display device, such as for example a cathode ray tube (CRT) or a liquid crystal display (LCD) or a light emitting diode (LED) monitor for displaying information to the user and a keyboard and a pointing device, such as for example a mouse or a trackball, by which the user may provide input to the computer. Other kinds of devices can be used to provide for interaction with a user as well. For example, feedback provided to the user can be any form of sensory feedback, such as for example visual feedback, auditory feedback, or tactile feedback; and input from the user may be received in any form, including acoustic, speech, or tactile input. Other possible input devices include touch screens or other touch-sensitive devices such as single or multi-point resistive or capacitive track pads, voice recognition hardware and software, optical scanners, optical pointers, digital image capture devices and associated interpretation software, and the like.

### VI. Example Definitions and Context

The disclosure is not limited to these exemplary embodiments and applications or to the manner in which the exemplary embodiments and applications operate or are described herein. Moreover, the figures may show simplified or partial views, and the dimensions of elements in the figures may be exaggerated or otherwise not in proportion.

Where reference is made to a list of elements (e.g., elements a, b, c), such reference is intended to include any one of the listed elements by itself, any combination of less than all of the listed elements, and/or a combination of all of the listed elements. Section divisions in the specification are for ease of review only and do not limit any combination of elements discussed.

Unless otherwise defined, scientific and technical terms used in connection with the present teachings described herein shall have the meanings that are commonly understood by those of ordinary skill in the art. Further, unless otherwise required by context, singular terms shall include pluralities and plural terms shall include the singular. Generally, nomenclatures utilized in connection with, and techniques of, chemistry, biochemistry, molecular biology, pharmacology, and toxicology are described herein are those well-known and commonly used in the art.

In addition, as the terms “on,” “attached to,” “connected to,” “coupled to,” or similar words are used herein, one element (e.g., a component, a material, a layer, a substrate, etc.) can be “on,” “attached to,” “connected to,” or “coupled to” another element regardless of whether the one element is directly on, attached to, connected to, or coupled to the other element or there are one or more intervening elements between the one element and the other element. In addition, where reference is made to a list of elements (e.g., elements a, b, c), such reference is intended to include any one of the listed elements by itself, any combination of less than all of the listed elements, and/or a combination of all of the listed elements. Section divisions in the specification are for ease of review only and do not limit any combination of elements discussed.

The term “subject” may refer to a subject of a clinical trial, a person or animal undergoing treatment, a person or animal undergoing anti-cancer therapies, a person or animal being monitored for remission or recovery, a person or animal undergoing a preventative health analysis (e.g., due to their medical history), or any other person or patient or animal of interest. In various cases, “subject” and “patient” may be used interchangeably herein.

The term “OCT image” or “OCTA image” may refer to an image of a tissue, an organ, etc., such as a retina, that is scanned or captured using optical coherence tomography (OCT) imaging technology and/or optical coherence tomography angiography (OCTA). The term may refer to one or both of 2D “slice” images and 3D “volume” images. When not explicitly indicated, the term may be understood to include OCT volume images.

Unless otherwise defined, scientific and technical terms used in connection with the present teachings described herein shall have the meanings that are commonly understood by those of ordinary skill in the art. Further, unless otherwise required by context, singular terms shall include pluralities and plural terms shall include the singular. Generally, nomenclatures utilized in connection with, and techniques of, chemistry, biochemistry, molecular biology, pharmacology, and toxicology are described herein are those well-known and commonly used in the art.

As used herein, “substantially” means sufficient to work for the intended purpose. The term “substantially” thus allows for minor, insignificant variations from an absolute or perfect state, dimension, measurement, result, or the like such as would be expected by a person of ordinary skill in the field but that do not appreciably affect overall performance. When used with respect to numerical values or parameters or characteristics that can be expressed as numerical values, “substantially” means within ten percent.

As used herein, the term “about” used with respect to numerical values or parameters or characteristics that can be expressed as numerical values means within ten percent of the numerical values. For example, “about 50” means a value in the range from 45 to 55, inclusive. The term “ones” means more than one.

As used herein, the term “plurality” can be 2, 3, 4, 5, 6, 7, 8, 9, 10, or more.

As used herein, the term “set of” means one or more. For example, a set of items includes one or more items.

As used herein, the phrase “at least one of,” when used with a list of items, means different combinations of one or more of the listed items may be used and only one of the items in the list may be needed. The item may be a particular object, thing, step, operation, process, or category. In other words, “at least one of” means any combination of items or number of items may be used from the list, but not all of the items in the list may be required. For example, without limitation, “at least one of item A, item B, or item C” means item A; item A and item B; item B; item A, item B, and item C; item B and item C; or item A and C. In some cases, “at least one of item A, item B, or item C” means, but is not limited to, two of item A, one of item B, and ten of item C; four of item B and seven of item C; or some other suitable combination.

As used herein, a “model” may include one or more algorithms, one or more mathematical techniques, one or more machine learning (ML) algorithms, or a combination thereof.

As used herein, “machine learning” may include the practice of using algorithms to parse data, learn from it, and then make a determination or prediction about something in the world. Machine learning uses algorithms that can learn from data without relying on rules-based programming.

As used herein, an “artificial neural network” or “neural network” may refer to mathematical algorithms or computational models that mimic an interconnected group of artificial neurons that processes information based on a connectionistic approach to computation. Neural networks, which may also be referred to as neural nets, can employ one or more layers of nonlinear units to predict an output for a received input. Some neural networks include one or more hidden layers in addition to an output layer. The output of each hidden layer is used as input to the next layer in the network, i.e., the next hidden layer or the output layer. Each layer of the network generates an output from a received input in accordance with current values of a respective set of parameters. In the various embodiments, a reference to a “neural network” may be a reference to one or more neural networks.

A neural network may process information in, for example, two ways; when it is being trained (e.g., using a training dataset) it is in training mode and when it puts what it has learned into practice (e.g., using a test dataset) it is in inference (or prediction) mode. Neural networks may learn through a feedback process (e.g., backpropagation) which allows the network to adjust the weight factors (modifying its behavior) of the individual nodes in the intermediate hidden layers so that the output matches the outputs of the training data. In other words, a neural network may learn by being fed training data (learning examples) and eventually learns how to reach the correct output, even when it is presented with a new range or set of inputs.

A neural network may process information in, for example, two ways; when it is being trained it is in training mode and when it puts what it has learned into practice it is in inference (or prediction) mode. Neural networks learn through a feedback process (e.g., backpropagation) which allows the network to adjust the weight factors (modifying its behavior) of the individual nodes in the intermediate hidden layers so that the output matches the outputs of the training data. In other words, a neural network learns by being fed training data (learning examples) and eventually learns how to reach the correct output, even when it is presented with a new range or set of inputs. A neural network may include, for example, without limitation, at least one of a Feedforward Neural Network (FNN), a Recurrent Neural Network (RNN), a Modular Neural Network (MNN), a Convolutional Neural Network (CNN), a Residual Neural Network (ResNet), an Ordinary Differential Equations Neural Networks (neural-ODE), or another type of neural network.

As used herein, “deep learning” may refer to the use of multi-layered artificial neural networks to automatically learn representations from input data such as images, video, text, etc., without human provided knowledge, to deliver highly accurate predictions in tasks such as object detection/identification, speech recognition, language translation, etc.

### VII. Recitation of Example Embodiments

Embodiment 1: A method, comprising: receiving a three-dimensional optical coherence tomography angiography (OCTA) volume of a retina of a subject, the OCTA volume comprising a plurality of layers; forming a slab input for a model system using the OCTA volume, the model system comprising a deep learning model; and generating, via the model system, a set of mask images based on the slab input, the set of mask images includes an area mask image that identifies an area of a foveal avascular zone captured by the OCT volume.

Embodiment 2. The method of embodiment 1, wherein the set of mask images further includes a boundary mask image that identifies a boundary of the foveal avascular zone.

Embodiment 3. The method of embodiment 1 or 2, wherein the set of mask images further includes a vessel mask image that identifies a plurality of vessels in the retina outside of the foveal avascular zone.

Embodiment 4. The method of any one of embodiments 1-3, further comprising: generating a set of foveal avascular zone (FAZ) measurements using at least one mask image of the set of mask images, wherein the set of FAZ measurements includes at least one of a first measurement for the area of the foveal avascular zone, a second measurement indicating a circularity of the foveal avascular zone, a third measurement indicating a tortuosity of the foveal avascular zone, a vessel density, a perfusion density, a fractal dimension, a vessel tortuosity index, or a vessel caliber index.

Embodiment 5. The method of any one of embodiments 1-4, further comprising, wherein generating the output comprises: generating an output based on the set of mask images, the output including an indication of a prognosis for the subject with respect to a retinal disease.

Embodiment 6. The method of embodiment 1, wherein forming the slab input comprises: processing the OCTA volume to identify an inner retinal slab comprised of a portion of the plurality of layers in the OCTA volume.

Embodiment 7. The method of embodiment 6, wherein forming the slab input further comprises: performing at least one preprocessing operation with respect to the inner retinal slab to form the slab input.

Embodiment 8. The method of any one of embodiments 1-7, wherein the model system includes a segmentation backbone, a foveal avascular zone module, a foveal avascular zone boundary module, a vessel module, and an output module and further comprising: training the model system to generate the set of mask images using a loss function that combines a loss metric for the foveal avascular zone module and at least one of the foveal avascular zone boundary module or the vessel module.

Embodiment 9. The method of embodiment 8, wherein the loss function includes a weighted Hausdorff distance.

Embodiment 10. The method of any one of embodiments 1-9, wherein the slab input is a three-dimensional volume identified from the OCTA volume in which the three-dimensional volume includes one or more plexuses that include at least one of a retinal nerve fiber layer plexus, a superficial vascular complex, a superficial capillary plexus, an intermediate capillary plexus, a deep capillary plexus, an outer retina plexus, a choriocapillaris plexus, or a choroid plex.

Embodiment 11. A method for training a model system, the method comprising:

receiving a training dataset that includes a plurality of optical coherence tomography angiography (OCTA) volumes for a plurality of retinas; performing a set of augmentation operations to form a 2D image input that includes a plurality of 2D images; and training a model system to generate a set of mask images that includes an area mask image that identifies an area of a foveal avascular zone using the two-dimensional training image input.

Embodiment 12. The method of embodiment 11, wherein the training includes: computing a loss using a loss function after each training cycle; and repeating the step of performing the set of augmentation operations for each next training cycle that is performed until the loss is minimized to within selected tolerances.

Embodiment 13. The method of embodiment 11 or embodiment 12, wherein performing the set of augmentation operations includes identifying a plurality of slabs based on the plurality of OCTA volumes.

Embodiment 14. The method of any one of embodiments 11-13, wherein performing the set of augmentation operations includes at least one of: performing a normalization operation for the plurality of OCT volumes; performing geometric augmentation, wherein the geometric augmentation includes at least one of a scaling operation, a resizing operation, a horizontal flipping operation, a vertical flipping operation, a cropping operation, or a rotation operation; pr performing slab augmentation.

Embodiment 15. The method of any one of embodiments 11-14, wherein performing the set of augmentation operations includes: performing projection augmentation that projects a three-dimensional slab onto a two-dimensional image in which an intensity value for each pixel in the two-dimensional image is determined as a maximum intensity value, an average intensity value, a minimum intensity value, a sum intensity value, a median intensity value, or a standard deviation intensity value of a set of intensity values associated with a corresponding stack of pixels in the three-dimensional slab.

Embodiment 16. The method of any one of embodiments 11 to 15, wherein training the model system includes training the model system to generate at least one of a boundary mask image and a vessel mask image based on the 2D image input.

Embodiment 17. The method of any one of embodiments 11 to 16, wherein the model system comprises a foveal avascular zone module, a foveal avascular zone boundary module, and a vessel module and wherein training the model system comprises:

computing a loss using a loss function that includes a cross entropy for the foveal avascular zone module, a cross entropy for the foveal avascular zone boundary module, and a cross entropy for the vessel module.

Embodiment 18. The method of embodiment 17, wherein the loss is computing using a plurality of training mask images that include a plurality of training area mask images, a plurality of boundary mask images, and a plurality of vessel mask images.

Embodiment 19. The method of embodiment 18, wherein the plurality of training mask images is generated manually, wherein the plurality of boundary mask images is generated based on the plurality of training mask images, and wherein the plurality of vessel images is generated based on amplitude thresholding and a clustering machine learning algorithm.

Embodiment 20. The method of any one of embodiments 17-19, wherein the loss function further includes a distance metric computed for the foveal avascular zone boundary module based on a boundary mask image generated by the foveal avascular zone boundary module.

Embodiment 21. A system, comprising: at least one data processor; and at least one memory storing instructions, which when executed by the at least one data processor, cause the at least one data processor to: receive a training dataset that includes a plurality of optical coherence tomography angiography (OCTA) volumes for a plurality of retinas; and training a model system over a plurality of training cycles using a loss function to generate a set of mask images that includes an area mask image that identifies an area of a foveal avascular zone based on a slab input. The model system comprises a foveal avascular zone module, a foveal avascular zone boundary module, and a vessel module. The loss function includes at least one loss metric for each of the foveal avascular zone module, the foveal avascular zone boundary module, and the vessel module.

Embodiment 22. A system, comprising: at least one data processor; and at least one memory storing instructions, which when executed by the at least one data processor, result in operations comprising the method of any of embodiments 1 to 20.

Embodiment 23. A non-transitory computer readable medium storing instructions, which when executed by at least one data processor, result in operations comprising the method of any of embodiments 1 to 20.

### Recitation of Alternative Example Embodiments

Embodiment A1. A computer-implemented method including generating, based at least on a three-dimensional volume including an optical coherence tomography angiography (OCTA) scan of a patient, a two-dimensional image of at least a portion of a retina of the patient; applying a segmentation model trained to identify, within the two-dimensional image, a foveal avascular zone (FAZ), a foveal avascular zone boundary, and a plurality of vessels; determining, based at least on the foveal avascular zone (FAZ), the foveal avascular zone boundary, and the plurality of vessels identified within the two-dimensional image, a first characteristic of the foveal avascular zone; and determining, based at least on the characteristic of the foveal avascular zone and/or the plurality of vessels, at least one of a disease progression and a treatment response for the patient.

Embodiment A2. The method of embodiment A1, wherein the first characteristic includes at least one of an irregularity of the foveal avascular zone and an enlargement of the foveal avascular zone.

Embodiment A3. The method of any one of embodiments A1 to A2, further including determining, based at least on the foveal avascular zone (FAZ), the foveal avascular zone boundary, and the plurality of vessels identified within the two-dimensional image, a second characteristic of the plurality of vessels.

Embodiment A4. The method of embodiment A3, wherein the second characteristic of the plurality of vessels include a capillary loss.

Embodiment A5. The method of any one of embodiments A1 to A4, wherein the segmentation model is a multi-headed machine learning model having a backbone network coupled to a first head trained to perform a first task of identifying the foveal avascular zone, a second head trained to perform a second task of identifying the foveal avascular zone boundary, and a third head trained to perform a third task of identifying the plurality of vessels.

Embodiment A6. The method of any one of embodiments A1 to A5, wherein the segmentation model identifies the foveal avascular zone by at least identifying, within the two-dimensional image, a foveal avascular zone (FAZ) mask including a plurality of pixels corresponding to the foveal avascular zone (FAZ).

Embodiment A7. The method of any one of embodiments A1 to A6, wherein the segmentation model identifies the foveal avascular zone boundary by at least identifying, within the two-dimensional image, a foveal avascular zone boundary mask including a plurality of pixels corresponding to the foveal avascular zone boundary.

Embodiment A8. The method of any one of embodiments A1 to A7, wherein the segmentation model identifies the plurality of vessels by at least identifying, within the two-dimensional image, a vessel mask including a plurality of pixels corresponding to the plurality of vessels.

Embodiment A9. The method of any one of embodiments A1 to A8, wherein the two-dimensional image is generated by at least projecting, onto a two-dimensional plane, the three-dimensional volume including the optical coherence tomography angiography (OCTA) scan of the patient.

Embodiment A10. The method of embodiment A9, wherein the projecting of the three-dimensional volume includes determining, for each pixel in the two-dimensional image, an intensity value.

Embodiment A11. The method of embodiment A10, wherein the intensity value of each pixel in the two-dimensional image is determined based at least on a set of intensity values associated within a corresponding stack of pixels in the three-dimensional volume.

Embodiment A12. The method of embodiment A11, wherein the set of intensity values includes, for each pixel within the stack of pixels, an intensity value corresponding to whether the pixel depicts a vessel in the retina of the patient.

Embodiment A13. The method of any one of embodiments A11 to A12, wherein the intensity value determined for each pixel in the two-dimensional image includes a maximum intensity value, an average intensity value, a minimum intensity value, a sum intensity value, a median intensity value, or a standard deviation intensity value of the set of intensity values associated with the corresponding stack of pixels in the three-dimensional volume.

Embodiment A14. The method of any one of embodiments A1 to A13, wherein the three-dimensional volume including the optical coherence tomography angiography (OCTA) scan of the patient includes one or more plexuses.

Embodiment A15. The method of embodiment A14, wherein the one or more plexuses include a retinal nerve fiber layer plexus, a superficial vascular complex, a superficial capillary plexus, an intermediate capillary plexus, a deep capillary plexus, an outer retina plexus, a choriocapillaris plexus, and/or a choroid plexus.

Embodiment A16. A computer-implemented method, including generating a training dataset that includes a set of two-dimensional images depicting at least a portion of a retina, each two-dimensional image of the set of two-dimensional images corresponding to a three-dimensional volume including an optical coherence tomography angiography (OCTA) scan of the retina; training, based at least on the training dataset, a segmentation model to perform a first task of identifying a foveal avascular zone (FAZ) within a two-dimensional image of at least a portion of the retina, a second task of identifying a foveal avascular zone boundary within the two-dimensional image, and a third task of identifying a plurality of vessels within the two-dimensional image; and applying the trained segmentation model to identify, within one or more two-dimensional images depicting at least a portion of a patient's retina, the foveal avascular zone, the foveal avascular zone boundary, and the plurality of vessels.

Embodiment A17. The method of embodiment A16, wherein the generating of the training dataset includes augmenting the set of two-dimensional images by at least generating, based on a first two-dimensional image included in the set of two-dimensional images, a second two-dimensional image for inclusion in the set of two-dimensional images.

Embodiment A18. The method of embodiment A17, wherein the second two-dimensional image is generated by one or more of flipping the first two-dimensional image horizontally, flipping the first two-dimensional image vertically, and rotating the first two-dimensional image.

Embodiment A19. The method of any one of embodiments A17 to A18, wherein the first two-dimensional image includes a first projection of a three-dimensional volume in two-dimensional space, and wherein the second two-dimensional image includes a second projection of the three-dimensional volume in two-dimensional space.

Embodiment A20. The method of embodiment A19, wherein the first projection and the second projection each include a different one of a maximum intensity projection, an average intensity projection, a minimum intensity projection, a sum intensity projection, a median intensity projection, and a standard deviation intensity projection.

Embodiment A21. The method of any one of embodiments A17 to A20, wherein the first two-dimensional image includes a first projection of a first three-dimensional volume including a first slab of the retina, and wherein the second two-dimensional image includes a second projection of a second three-dimensional volume including a second slab of the retina.

Embodiment A22. The method of embodiment A21, wherein the first slab and the second slab includes one or more different layers of the retina.

Embodiment A23. The method of any one of embodiments A16 to A22, wherein the generating of the training dataset includes generating, for each two-dimensional image included in the set of two-dimensional images, a ground-truth annotation identifying a plurality of pixels depicting the foveal avascular zone.

Embodiment A24. The method of any one of embodiments A16 to A23, wherein the generating of the training dataset includes applying edge detection to generate, for each two-dimensional image included in the set of two-dimensional images, a ground-truth annotation identifying a plurality of pixels depicting the foveal avascular zone boundary.

Embodiment A25. The method of any one of embodiments A16 to A24, wherein the generating of the training dataset includes applying adaptive thresholding to generate, for each two-dimensional image included in the set of two-dimensional images, a ground-truth annotation identifying a plurality of pixels depicting the plurality of vessels.

Embodiment A26. The method of any one of embodiments A16 to A25, wherein the segmentation model is a multi-headed machine learning model having a backbone network coupled to a first head trained to perform the first task of identifying the foveal avascular zone, a second head trained to perform the second task of identifying the foveal avascular zone boundary, and a third head trained to perform the third task of identifying the plurality of vessels.

Embodiment A27. The method of embodiment A26, wherein the training of the segmentation model includes simultaneously training the first head of the segmentation model to perform the first task, the second head of the segmentation model to perform the second task, and the third head of the segmentation model to perform the third task.

Embodiment A28. The method of any one of embodiments A26 to A27, wherein the training of the segmentation model includes training the backbone network.

Embodiment A29. The method of any one of embodiments A16 to A28, wherein the training of the segmentation model includes simultaneously minimizing a first loss function associated with the first head of the segmentation model performing the first task, a second loss function associated with the second head of the segmentation model performing the second task, and a third loss function associated with the third head of the segmentation model performing the third task.

Embodiment A30. The method of embodiment A29, wherein the first loss function, the second loss function, and the third loss function include one or more of a distribution-based loss, a region-based loss, a boundary based loss, and a compounded loss.

Embodiment A31. The method of any one of embodiments A29 to A30, wherein one or more of the first loss function, the second loss function, and the third loss function are cross entropy loss functions quantifying a difference between a first probability distribution of labels assigned by the trained segmentation model to a plurality of pixels in the two-dimensional image and a second probability distribution of ground truth labels associated with the plurality of pixels in the two-dimensional image.

Embodiment A32. The method of any one of embodiments A29 to A31, wherein the second loss function includes a distance metric quantifying a distance between a first set of pixels identified by the second head as being a part of the foveal avascular zone boundary and a second set of pixels forming a corresponding ground truth foveal avascular zone boundary.

Embodiment A33. The method of embodiment A32, wherein the distance metric is a weighted Hausdorff distance to increase a sharpness of the foveal avascular zone boundary identified by the second head.

Embodiment A34. The method of any one of embodiments A16 to A33, wherein the training dataset is generated to include a first two-dimensional image corresponding to a first optical coherence tomography angiography (OCTA) scan produced by a first scanner and a second two-dimensional image corresponding to a second optical coherence tomography angiography (OCTA) scan produced by a second scanner.

Embodiment A35. The method of embodiment A34, wherein the first scanner is a swept-source scanner and the second scanner is a spectral domain scanner.

Embodiment A36. The method of any one of embodiments 34 to 35, wherein the generating of the training dataset includes normalizing the first two-dimensional image corresponding to the first optical coherence tomography angiography (OCTA) scan produced by the first scanner and the second two-dimensional image corresponding to the second optical coherence tomography angiography (OCTA) scan produced by the second scanner.

Embodiment A37. The method of any one of embodiments A16 to A36, wherein the training dataset is generated to include a first two-dimensional image corresponding to a first optical coherence tomography angiography (OCTA) scan that has been processed to remove motion artifacts and a second two-dimensional image corresponding to a second optical coherence tomography angiography (OCTA) scan that has not been processed to remove motion artifacts.

Embodiment A38. The method of any one of embodiments A16 to A37, wherein the training dataset is generated to include a first two-dimensional image corresponding to a first optical coherence tomography angiography (OCTA) scan that has undergone registration and merging to remove motion artifacts and a second two-dimensional image corresponding to a second optical coherence tomography angiography (OCTA) scan that has not undergone registration and merging to remove motion artifacts.

Embodiment A39. The method of any one of embodiments A16 to A38, wherein the training dataset is generated to include a plurality of two-dimensional images that correspond to different quality optical coherence tomography angiography (OCTA) scans produced by a same scanner.

Embodiment A40. The method of any one of embodiments A16 to A39, wherein the training dataset is generated to include a first two-dimensional image of a first retina exhibiting a first disease severity, a first disease burden, and/or a first disease prognosis, and wherein the training dataset is further generated to include a second two-dimensional image of a second retina exhibiting a second disease severity, a second disease burden, and/or a second disease prognosis.

Embodiment A41. A system, including at least one data processor; and at least one memory storing instructions, which when executed by the at least one data processor, result in operations including the method of any of embodiments A1 to A40.

Embodiment A42. A non-transitory computer readable medium storing instructions, which when executed by at least one data processor, result in operations including the method of any of embodiments A1 to A40.

### VIII. Additional Considerations

The headers and subheaders between sections and subsections of this document are included solely for the purpose of improving readability and do not imply that features cannot be combined across sections and subsection. Accordingly, sections and subsections do not describe separate embodiments.

Some embodiments of the present disclosure include a system including one or more data processors. In some embodiments, the system includes a non-transitory computer readable storage medium containing instructions which, when executed on the one or more data processors, cause the one or more data processors to perform part or all of one or more methods and/or part or all of one or more processes disclosed herein. Some embodiments of the present disclosure include a computer-program product tangibly embodied in a non-transitory machine-readable storage medium, including instructions configured to cause one or more data processors to perform part or all of one or more methods and/or part or all of one or more processes disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention as claimed has been specifically disclosed by embodiments and optional features, modification and variation of the concepts herein disclosed may be resorted to by those skilled in the art, and that such modifications and variations are considered to be within the scope of this invention as defined by the appended claims.

While the present teachings are described in conjunction with various embodiments, it is not intended that the present teachings be limited to such embodiments. On the contrary, the present teachings encompass various alternatives, modifications, and equivalents, as will be appreciated by those of skill in the art.

In describing the various embodiments, the specification may have presented a method and/or process as a particular sequence of steps. However, to the extent that the method or process does not rely on the particular order of steps set forth herein, the method or process should not be limited to the particular sequence of steps described, and one skilled in the art can readily appreciate that the sequences may be varied and still remain within the spirit and scope of the various embodiments.

Further, the subject matter described herein can be embodied in systems, apparatus, methods, and/or articles depending on the desired configuration. The implementations set forth in the description herein do not represent all implementations consistent with the subject matter described. Instead, they are merely some examples consistent with aspects related to the described subject matter. Although a few variations have been described in detail above, other modifications or additions are possible. In particular, further features and/or variations can be provided in addition to those set forth herein. For example, the implementations described above can be directed to various combinations and sub-combinations of the disclosed features and/or combinations and sub-combinations of several further features disclosed above. In addition, the logic flows depicted in the accompanying figures and/or described herein do not necessarily require the particular order shown, or sequential order, to achieve desirable results. Other implementations may be within the scope of the following claims.

What is claimed is:

1. A method, comprising:
receiving a three-dimensional optical coherence tomography angiography (OCTA) volume of a retina of a subject, the OCTA volume comprising a plurality of layers;
forming a slab input for a model system using the OCTA volume, the model system comprising a deep learning model; and
generating, via the model system, a set of mask images based on the slab input, the set of mask images includes an area mask image that identifies an area of a foveal avascular zone captured by the OCTA volume.

2. The method of claim 1, wherein the set of mask images further includes at least one of:
a boundary mask image that identifies a boundary of the foveal avascular zone; or
a vessel mask image that identifies a plurality of vessels in the retina outside of the foveal avascular zone.

3. The method of claim 1, further comprising:
generating a set of foveal avascular zone (FAZ) measurements using at least one mask image of the set of mask images, wherein the set of FAZ measurements includes at least one of a first measurement for the area of the foveal avascular zone, a second measurement indicating a circularity of the foveal avascular zone, a third measurement indicating a tortuosity of the foveal avascular zone, a vessel density, a perfusion density, a fractal dimension, a vessel tortuosity index, or a vessel caliber index.

4. The method of claim 1, further comprising generating an output based on the set of mask images, the output including an indication of a prognosis for the subject with respect to a retinal disease.

5. The method of claim 1, wherein forming the slab input comprises:
processing the OCTA volume to identify an inner retinal slab comprised of a portion of the plurality of layers in the OCTA volume.

6. The method of claim 5, wherein forming the slab input further comprises:
performing at least one preprocessing operation with respect to the inner retinal slab to form the slab input.

7. The method of claim 1,
wherein the model system includes a segmentation backbone, a foveal avascular zone module, a foveal avascular zone boundary module, a vessel module, and an output module, and
wherein the method further comprises training the model system to generate the set of mask images using a loss function that combines a loss metric for the foveal avascular zone module and at least one of the foveal avascular zone boundary module or the vessel module.

8. The method of claim 7, wherein the loss function includes a weighted Hausdorff distance.

9. The method of claim 1, wherein the slab input is a three-dimensional volume identified from the OCTA volume in which the three-dimensional volume includes one or more plexuses that include at least one of a retinal nerve fiber layer plexus, a superficial vascular complex, a superficial capillary plexus, an intermediate capillary plexus, a deep capillary plexus, an outer retina plexus, a choriocapillaris plexus, or a choroid plex.

10. A method for training a model system, the method comprising:
receiving a training dataset that includes a plurality of optical coherence tomography angiography (OCTA) volumes for a plurality of retinas;
performing a set of augmentation operations to form a 2D training image input that includes a plurality of 2D images; and
training a model system to generate a set of mask images that includes an area mask image that identifies an area of a foveal avascular zone using the 2D training image input.

11. The method of claim 10, wherein the training includes:
computing a loss using a loss function after each training cycle; and
repeating the step of performing the set of augmentation operations for each next training cycle that is performed until the loss is minimized to within selected tolerances.

12. The method of claim 10, wherein performing the set of augmentation operations includes identifying a plurality of slabs based on the plurality of OCTA volumes.

13. The method of claim 10, wherein performing the set of augmentation operations includes at least one of:
performing a normalization operation for the plurality of OCTA volumes;
performing geometric augmentation, wherein the geometric augmentation includes at least one of a scaling operation, a resizing operation, a horizontal flipping operation, a vertical flipping operation, a cropping operation, or a rotation operation; or
performing slab augmentation.

14. The method of claim 10, wherein performing the set of augmentation operations includes:
performing projection augmentation that projects a three-dimensional slab onto a two-dimensional image in which an intensity value for each pixel in the two-dimensional image is determined as a maximum intensity value, an average intensity value, a minimum intensity value, a sum intensity value, a median intensity value, or a standard deviation intensity value of a set of intensity values associated with a corresponding stack of pixels in the three-dimensional slab.

15. The method of claim 10, wherein training the model system includes training the model system to generate at least one of a boundary mask image and a vessel mask image based on the 2D image input.

16. The method of claim 10,
wherein the model system comprises a foveal avascular zone module, a foveal avascular zone boundary module, and a vessel module; and
wherein training the model system comprises computing a loss using a loss function that includes a cross entropy for the foveal avascular zone module, a cross entropy for the foveal avascular zone boundary module, and a cross entropy for the vessel module.

17. The method of claim 16, wherein the loss is computing using a plurality of training mask images that include a plurality of training area mask images, a plurality of boundary mask images, and a plurality of vessel mask images.

18. The method of claim 17, wherein the plurality of training mask images is generated manually, wherein the plurality of boundary mask images is generated based on the plurality of training mask images, and wherein the plurality of vessel mask images is generated based on amplitude thresholding and a clustering machine learning algorithm.

19. The method of claim 18, wherein the loss function further includes a distance metric computed for the foveal avascular zone boundary module based on a boundary mask image generated by the foveal avascular zone boundary module.

20. A system, comprising:
at least one data processor; and
at least one memory storing instructions, which when executed by the at least one data processor, cause the processor to:
receive a training dataset that includes a plurality of optical coherence tomography angiography (OCTA) volumes for a plurality of retinas; and
training a model system over a plurality of training cycles using a loss function to generate a set of mask images that includes an area mask image that identifies an area of a foveal avascular zone based on a slab input, wherein the training comprises:
wherein the model system comprises a foveal avascular zone module, a foveal avascular zone boundary module, and a vessel module; and
wherein the loss function includes at least one loss metric for each of the foveal avascular zone module, the foveal avascular zone boundary module, and the vessel module.
